SEQUENCE LISTING (53 sequences)

SEQ ID NO: 1 - DNA, 1321 bp, Human
caacaatcgggatataaacccaggcattcgagctggcaacagcagcccccctttgggtcccttccctttgtatgggagctgttttcatgctatttcactctattaaatcttgcaactgcactcttctggtccatgtttcttacggctcgagctgagcttttgctcaccgtccaccactgctgtttgccaccaccgcagacctgccgctgactcccatccctctggatcctgcagggtgtccgctgtgctcctgatccagcgaagcgcccattgccgctcccaattgggctaaaggcttgccattgttcctgcacggctaagtgcctgggtttgttctaattgagctgaacactagtcactgggttccatggttctcttctgtgacccacggcttctaatagaactataacacttaccacatggcccaagattccattccttggaatccgtgaggccaagaactccaggtcagagaatacgaagcttgccaccatcttggaagcggcctgctaccatcttggaagtggttcaccaccatcttgggagctctgtgagcaaggaccccccggtaacattttggcaaccacgaacggacatccaaagtgatgggaaacgttccccgcaagacaaaaacgcccctaagacgtattctggaaaattgggaacaatttgaccctcagacactaagaaagaaacgacttatattcttctgcagtgccgcctggcactcctgagggaagtataaattataacaccatcttacagctagacctcttttgtagaaaaggcaaatggagtgaagtgccataagtacaaactttcttttcattaagagacaactcacaattatgtaaaaagtgtgatttatgccctacaggaagccttcagagtctacctccctatcccagcatccccgactccttccccacttaataaggaccccccttcaacccaaatggtccaaaaggagatagacaaaagggtaaacagtgaaccaaagagtgccaatattccccaattatgacccctccaagcagtgggaggaagagaattcggcccagccagagtgcatgtgcctttttctctcccagacttaaagcaaataaaaacagacttaggtaaattctcagataaccctgatggctatattggtgttttacaagggttaggacaattctttgatctgacatggagagatatatatgtcactgctaaatcagacactaaccccaaatgagagaagtgccaccataactgcagcctgagagtttggcgatctctggtatctcagtcaggtcaatgataggatgacaacagagga

SEQ ID NO: 2 - DNA, 2938 bp, Human
caacgacggacatccaaagtgatgggaaacgttccccgcaagacaaaaacgcccctaagacgtattctggagaattgggaccaatttgaccctcagacactaagaaagaaacgacttatattcttctgcagtgccgcctggcactcctgagggaagtataaattataacaccatcttacagctagacttcttttgtagaaaaggcaaatggagtgaagtgccataagtacaaactttcttttcattaagagacaactcacaattatgtaaaaagtgtgatttatgccctacaggaagccttcagagtctacctccctatcccagcatccccgactccttccccaactaataaggaccccccttcaacccaaatggtccaaaaggagatagacaaaagggtaaacagtgaaccaaagagtgccaatattccccaattatgacccctcccaagcagtgggaggaagagattcggcccagccagagtgcatgtgctttttcttctcccagacttaaagcaaataaaaacagacttaggtaaattctcagataatcctgatggctatattgatgttttacaagggttaggacaattctttgatctgacatggagagatataatgtcactgctaaatcagacactaaccccaaatgagagaagtgccaccataactgcagcctgagagtttggcgatctctggtatctcagtcaggtcaatgataggatgacaacagaggaaagagatgatccccacagccagcaagcagttcccagtctasaccctcattggggacacagaaatcagtaacatgggagattggtgctgcagacatttgctaacttgtgtgctacaaggactaaggaaaactacgaagaaaatctacgaattactcaatgatgtccaccataacacaggggaagggaagaaaatcctactgcctttctggagagactaagggaggcattgaggaagcgtgcctctctgtcacctgactcttctgaaggccaactaatcttaaagcgtaagtttatcactcagtcagctgcagacattagaaaaaacttcaaaagtctgccgtaggcccggagcaaaacttagaaaccctattgaacttggcaacytcggttttttataatagagatcaggaggagcaggcggaacaggacaaacgggattaaaaaaaaggccaccgctttagtcatgaccctcaggcaagtggactttggaggctctggaaaagggaaaagctgggcaaattgaatgcctaatagggcttgcttccagtgcggtctacaaggacactttaaaaaagattgtccaagtagaagtaagccgccccttcgtccatgccccttatttcaagggaatcactggaaggcccactgccccaggggacaaaggtcttttgagtcagaagccactaaccagatgatccagcagcaggactgagggtgcctggggcaagcgccatcccatgccatcaccctcacagagccctgggtatgcttgaccattgagggccaggaaggttgtctcctggacactggtgcggtcttcttagtcttactcttctgtcccggacaactgtcctccagatctgtcactatctgagggggtcctaagacgggcagtcactagatacttctcccagccactaagttatgactggggagctttattcttttcacatgcttttctaattatgcttgaaagccccactaccttgttagggagagacattctagcaaaagcaggggccattatacacctgaacataggagaaggaacacccgtttgttgtcccctgcttgaggaaggaattaatcctgaagtctgggcaacagaaggacaatatggacgagcaaagaatgcccgtcctgttcaagttaaactaaaggattccacttcctttccctaccaaaggcagtaccccctcagacccaaggcccaacaaggattccaaaagattgttaaggacttaaaagcccaaggcttagtaaaaccatgcataactccctgcagtaattccgtagtggattgaggaggcacagaaacccagtggacagtggagggttagtgcaagatctcaggattatcaatggaggccgttgtccttttatacccagctgtacctagcccttatactgtgctttcccaaataccagaggaagcagagtggtttacactcctggaccttaaggatgccttcttctgcatccctgtacatcctgactctcaattcttgtttgcctttgaagatacttcaaacccaacatctcaactcacctggactgttttaccccaagggttcagggatagcccccatctatttggccaggcattagcccaagacttgagccaatcctcatacctggacacttgtccttcggtaggtggatgatttacttttggccgcccattcagaaaccttgtgccatcaagccacccaagcgctcttcaatttcctcgctacctgtggctacatggtttccaaaccaaaggctcaactctgctcacagcaggttacttagggctaaaattatccaaaggcaccagggccctcagtgaggaacacatccagcctatactggcttatcctcatcccaaaaccctaaagcaactaaggggattccttggcgtaataggtttctgccgaaaatggattcccaggtttggcgaaatagccaggtcattaaatacactaattaaggaaactcagaaagccaatacccatttagtaagatggacaactgaagtagaagtggctttccaggccctaacccaagccccagtgttaagtttgccaacagggcaagacttttcttcatatgtcacagaaaaaacaggaatagctctaggagtccttacacagatccgagggatgagcttgcaacctgtggcgtacctgactaaggaaattgatgtagtggcaaagggtt

SEQ ID NO: 3 - DNA, 1422 bp, Human
  misc_feature (879)..(879): n = any nucleotide
tcagggatagcccccatctatttggccaggcattagcccaagacttgagtcagttatcatacctggacactcttgtccttcagtatgtggatgatttacttttagctgcctgttcagaaaccttgtgccatcaagccacccaagcactcttaaatttcctcgccacctgtggctacaaggtttccaaagagaagctcagctctgctcacagcaggttaaatacttaggactaagattatccaaaggcaccaaggccctcagtgaggaatgtatccagcctatactggcttatcctcatctcaaaaccctaaagcaactaagagagttccttggcataacaggcttctgccgaatatggattccccaggtatggcaaaatagccaggccattatatacagtaattaaggaaactcagaaagccaatacccatttaataagatggatacctgaagccaaagtggctttccaggcccctaaagaaggccttaaacccaagtcccagtgttaagcttgccaacggggcaagacttttctttatacatcacagaaaaaaacagaaacagctctgggagtccttacacaggtccaagggacgagcttgcaacccatggcatacctgagtaaggaaactgatgtagtggcaaagggttggcttcattgtttatgggtagtggtggcagtagcagttgtagtatctgaagcagttaaaataatacaggggagagatcttactgtgtggacatctcatgaggtgaacagcatactcactgctaaaggagacttgtggctgtcagacaaccgtttacttaaatatcaggctctattacttgaaaggccagtgctgcaactgtgcacttgtgcaactcttaacccagtcncatttcttccagacaatgaagatagaatataactgtcaacaaataatttctcaaacctatgccactcgaggggaccttctagaagttcccttgactgatcctgaccttcaacttgtatactgatggaagttcctttgtagaaaaaggacttcaaaagcggggtatgcagtggtcagtgataatggaatatttgaaagtatcccctcactccaggaactagtgcttagctggcagaactaatagccttcattggggcactagaattaggagaaggaaaaagggtaaatatatatacagactctgagtatgctcacctagtcntccatgcccatgaggcaatatgcagagaaagggaattcctaacttccgagggaacacctatcacacatcaggaagccattaggagattattactggcagtacagaaacctaaagaggtggaagtcttacactgctggggtcatcagaaaggaaagaaaagggaaatagaagggaattgccaagcagatattgaagcaaaaagagctgcaaggcaggaccctc

SEQ ID NO: 4 - DNA, 2006 bp, Human
  misc_feature (305)..(305): n = any nucleotide
atgcagtggtcagtgataatggaatacttgaaagtaatcccctcactccaggaactagtgctcagctagcagaactaatagccctcacttgggcactagaattaggagaagaaaaaagggcaaatatatatacagactctaaatatgcttacctagtcctccatgcccatgcagcaatatggaaagaaagggaattcctaacttctgagagaacacctatcaaacatcaggaagccattaggaaattattattggctgtacagaaacctaaagaggtggcagtcttacactgccggggtcatcanaaaggaaaggaaagggaaaatacttttgcctgcaactatccaatggaaattacttaaaacccttcatcaaacctttcacttaggcatcgatagcacccatcaaatggccaaatcattatttactggaccaggccttttcaaaactatcaagcaaatattcagggcctgtgaattgtgccaaaaaaataatcccctgcctcatcgccaagctccttcaggaaaacaaaaaacaggccattaccctgaaaaaaactggcaactgattttacccacaagcccaaacctcagggatttcagtatctactagtctgggtaaatactttcacgggttgggcaaaggccttcccctgtaggacagaaaaggcccaagaggtaataaaggcactagttcatgaaataattcccagattcggacttccccgaggcttacagagtgacaatagccctgctttccaggccacagtaacccagggagtatcccaggcgttaggtatacgatatcacttacactgcgcctgaaggccacagtcctcagggaaggtcgagaaaatgaatgaaatactcaaaggacatctaaaaaagcaaacccaggaaacccacctcacatggcctgctctgttgcctatagccttaaaaagaatctgcaactttccccaaaaagcaggacttagcccatacgaaatgctgtatggaaggcccttcataaccaatgaccttgtgcttgacccaagacagccaacttagttgcagacatcacctccttagccaaatatcaacaagttcttaaaacattacaaggaacctatccctgagaagagggaaaagaactattccacccttgtgacatggtattagtcaagtcccttctctctaattccccatccctagatacatcctgggaaggaccctacccagtcattttatttaccccaactgcggttaaagtggctggagtggtcttggatacatcacacttgagtcaaatcctggatactgccaaaggaacctgaaaatccaggagacaacgctagctattcctgtgaacctctagaggatttgcgcctgctcttcaaacaacaaccaggaggaaagtaactaaaatcataaatcccccatggccctcccttatcatatttttctctttactgttcttttaccctctttcactctcactgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaagcca

SEQ ID NO: 5 - DNA, 1948 bp, Human
  misc_feature (84)..(84): n = any nucleotide
actgcactcttctggtccatgtttcttacggctcgagctgagcttttgctcaccgtccaccactgctgtttgccaccaccgcanacctgccgctgactcccatccctctggatcctgcagggtgtccgctgtgctcctgatccagcgaggcgcccattgccgctcccaattgggctaaaggcttgccattgtncctgcacggctaagtgcctgggtttgttctaattgagctgaacactantcactgggttccatggttctcttctgtgacccacggcttctaatagaactataacacttaccacatggcccaagattccattccttggaatccgtgagggcaagaactccaggtcagagaatacgaggcttgccaccatcttggaagcggcctgctaccatcttggaagtggttcaccaccatcttgggagctctgtgagcaaggaccccccggtaacattttggcaaccacgaacggacatccaaagtgatacatcctgggaaggaccctacccagtcattttatctaccccaactgcggttaaagtggctggagtggagtcttggatacatcacacttgagtcaaatcctggatactgccaaaggaacctgaaaatccaggagacaacgctagctattcctgtgaacctctagaggatttgcgcctgctcttcaaacaacaaccaggaggaaagtaactaaaatcataaatccccatggccctcccttatcatatttttctctttactgttgtttcaccctctttcactctcactgcaccccctccatgccgctgtatgaccagtagctccccttaccaagagtttctatggagaatgcagcgtcccggaaatattgatgccccatcgtataggagtctttgtaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatggccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaaagagggggaacctgtttatttttaggggaagaatgctgttattatgttaatcaa

SEQ ID NO: 6 - DNA, 1136 bp, Human
ccatggccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagactcgctaaccgctgaaagagggggaacctgtttatttttaggggaagaatgctgttattatgttaatcaatccggaatcgtcactgagaaagttaaagaaattcgagatcgaatacaacgtagagcagaagagcttcgaaacactggaccctggggcctcctcagccaatggatgccctggattctccccttcttaggacctctagcagctataatattgctactcctctttggaccctgtatctttaacctccttgttaactttgtctcttccagaatcgaagctgtaaaactacaaatggagcccaagatgcagtccaagactaagatctaccgcagacccctggaccggcctgctagcccacgatctgatgttaatgacatcaaaggcacccctcctgaggaaatctcagctgcacaacctctactacgccccaattcagcaggaagcagttagagcggtcgtcggccaacctccccaacagcacttaggttttcctgttgagatgggggactgagagacaggactagctggatttcctaggctgactaagaatccctaagcctagctgggaaggtgaccacatccacctttaaacacggggcttgcaacttagttcacacctgaccaatcagagagctcactaaaatgctaattaggcaaagacaggaggtaaagaaatagccaatcatctattgcatgagagcacagcaggagggacaatgatcgggatataaacccaagtcttcgagccggcaacggcaaccccctttgggtcccctccctttgtatgggagctctgttttcatgctatttcactctattaaatcttgcagctgcgaaaaaaaaaaaaaaaaaaaa

SEQ ID NO: 7 - DNA, 2782 bp, Human
atgggagctgttttcatgctatttcactctattaaatcttgcaactgcactcttctggtccatgtttcttacggctcgagctgagcttttgctcaccgtccaccactgctgtttgccaccaccgcagacctgccgctgactcccatccctctggatcctgcagggtgtccgctgtgctcctgatccagcgaagcgcccattgccgctcccaattgggctaaaggcttgccattgttcctgcacggctaagtgcctgggtttgttctaattgagctgaacactagtcactgggttccatggttctcttctgtgacccacggcttctaatagaactataacacttaccacatggcccaagattccattccttggaatccgtgaggccaacgaactccaggtcagagaatacgaagcttgccaccatcttggaagcggcctgctaccatcttggaagtggttcaccaccatcttgggagctctgtgagcaaggaccccccggtgacattttggcgaccaccaacggacatcccaagtgatacatcctgggaaggaccctacccagtcattttatctaccccaactgcggttaaagtggctggagtggagtcttggatacatcacacttgagtcaaatcctggatactgccaaaggaacctgaaaatccaggagacaacgctagctattcctgtgaacctctagaggatttgcgcctgctcttcaaacaacaaccaggaggaaagtaactaaaatcataaatccccatgggcctcccttatcatatttttctctgtagtgttctttcaccctgtttcactctcactgcaccccctccatgccgctgtatgaccagtagctcccctcacccagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcaccggggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatgtggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcgaaatcgaagagctttagacttgctaaccgctgagagagggggaacctgtttatttttaggggaagaatgctgttattatgttaatcaatccggaatcgtcactgagaaagttgaagaaattccagatcgaatacaacgtatagcagaggagcttcgaaacactggaccctggggcctcctcagccgatggatgccctggattctccccttcttaggacctctagcagctataatattgctactcctctttggaccctgtatctttgacctccttgttaactttgtctcttccagaatcgaagctgtgaaactacaaatggagcccaagatgcagtccaagactaagatctaccgcagacccctggaccggcctgctagcccacgatctgatgttaatgacatcaaaggcacccctcctgaggaaatctcagctgcacaacctctactacgccccaattcagcaggaagcagttagagcggtggtcggccaacctccccaacagcacttaggttttcctgttgagatgggggactgagagacaggactagctggatttcctaggctgactaagaatccttaagcctaggtgggaaggtgaccacatccacctttaaacacggggcttgcaacttagctcacacctgaccaatcagagagctcactaaaatgctaattaggcaaagacaggaggtaaagaaatagccaatcatttattgcctgagagcacagcaggagggacaatgatcgggatataaacccaagttttcgagccggcaacggcaaccccctttgggtcccctccctttgtatgggagctctgttttcatgctatttcactctattaaatcttgcaactgcaaaaaaaaaaaaaaaaaaa

SEQ ID NO: 8 - DNA, 666 bp, Human
  misc_feature (119)..(119): n = any nucleotide
tgtccgctgtgctcctgatccagcgaggcgcccattgccgctcccaattgggctaaaggcttgccattgttcctgcacggctaagtgcctgggtttgttctaattgagctgaacactantcactgggttccatggttctcttctgtgacccacggcttctaatataactataacacttaccacatggcccaagattccattccttggaatccgtgaggccaagaactccaggtcagagaatacgaggcttgccaccatcttggaagcggcctgctaccatcttggaagtggttcaccaccatcttgggagctctgtgagcaaggaccccccggtaacattttggcaaccacgaacggacatccaaagtgaatcgaagctgtaaaactacaaatggagcccaagatgcagtccaagactaagatctaccgcagacccctggaccggcctgctagcccacgatctgatgttaatgacatcaaaggcacccctcctgaggaaatctcagctgcacaacctctactacgccccaattcagcaggaagcagttagagcggtcgtcggccaacctccccaacagcacttaggttttcctgttgagatgggggactgagagacaggactagctggatttcctaggctgactaagaatccctaagcctagctgg

SEQ ID NO: 9 - DNA, 3372 bp, Human
gacttcccaaataccagaggaagcagagtggtttacagtcctggaccttcaggatgccttcttctgcatccctgtacatcctgactctcaattcttgtttgcctttgaagatacttcaaacccagcatctcaactcacctggactattttaccccaagggttcagggatagtccccatctatttggccaggcattagcccaagacttgagccaatcctcatacctggacacttgtccttcggtaggtggatgatttacttttggccgcccattcagaaaccttgtgccatcaagccacccaagcgctcttcaatttcctcgctacctgtggctacatggtttccaaaccaaaggctcaactctgctcacagcaggttacttagggctaaaattatccaaaggcaccagggccctcagtgaggaacacatccagcctatactggcttatcctcatcccaaaaccctaaagcaactaaggggattccttggcgtaataggtttctgccgaaaatggattcccaggtatggcgaaatagccaggtcattaaatacactaattaaggaaactcagaaagccaatacccatttagtaagatggacaactgaagtagaagtggctttccaggccctaacccaagccccagtgttaagtttgccaacagggcaagacttttgttcatatgtcacagaaaaaacaggaatagctctaggagtccttacacagatccgagggatgagcttgcaacctgtggcacacctgactaaggaaattgatgtagtggcaaagggttgacctcattgtttacgggtagtggtggcagtagcagtcttagtatctgaagcagttaaaataatacagggaagagatcttactgtgtggacatctcatgatgtgaatggcatactcactgctaaaggagacttgtggctgtcagacaactgtttacttaaatgtcaggctctattacttgaagggccagtgctgcgactgtgcacttgtgcaactcttaacccagccacatttcttccagacaatgaagaaaagataaaacataactgtcaacaagtaatttctcaaacctatgccactcgaggggaccttttagaggttcctttgactgatcccgacctcaacttgtatactgatggaagttcctttgtagaaaaaggacttcgaaaagtggggtatgcagtggtcagtgataatggaatacttgaaagtaatcccctcactccaggaactagtgctcagctagcagaactaatagccctcacttgggcactagaattaggagaagaaaaaagggcaaatataatacagactctaaatatgcttacctagtcctccatgcccatgcagcaatatggaaagaaagggaattcctaacttctgagagaacacctatcaaacatcaggaagccattaggaaattattattggctgtacagaaacctagagaggtggcagtcttacactgccggggtcatcacaaaggaaaggaaagggaaatacaagagaactgccaagcatatattgaagccaaaagagctgcaaggcaggaccctccattagaaatgcttattaaacttcccttagtatagggtaatcccttccgggaaaccaagccccagtactcagcaggagaaacagaatggggaacctcacgaggcagttttctcccctcgggacggttagccactgaagaagggaaaatacttttgcctgcaactatccaatggaaattacttaaaacccttcatcaaacctttcacttaggcatcgatagcacccatcagatggccaaatcattatttactggaccaggccttttcaaaactatcaagcagatagtcagggcctgtgaagtgtgccagagaaataatcccctgccttatcgccaagctccttcaggagaacaaagaacaggccattaccctggagaagactggcaactgattttacccacaagcccaaacctcagggatttcagtatctactagtctgggtagatactttcacgggttgggcagaggccttcccctgtaggacagaaaaggcccaagaggtaataaaggcactagttcatgaaataattcccagattcggacttccccgaggcttacagagtgacaatagccctgctttccaggccacagtaacccagggagtatcccaggcgttaggtatacgatatcacttacactgcgcctgaaggccacagtcctcagggaaggtcgagaaaatgaatgaaacactcaaaggacatctaaaaaagcaaacccaggaaacccacctcacatggcctgttctgttgcctatagccttaaaaagaatctgcaactttccccaaaaagcaggacttagcccatacgaaatgctgtatggaaggcccttcataaccaatgaccttgtgcttgacccaagacagccaacttagttgcagacatcacctccttagccaaatatcaacaagttcttaaaacattacaaggaacctatccctgagaagaggaaaagaatattccacccaagtgacatggtattagtcaagtcccttccctctaattccccatccctagatacatcctgggaaggaccctacccagtcattttatctaccccaactgcggttaaagtggctggagtggagtcttggatacatcacacttgagtcaaatcctggatactgccaaaggaacctgaaaatccaggagacaacgctagctattcctgtgaacctctagaggatttgcgcctgctcttcaaacaacaaccaggaggaaaaatcgaagctgtaaaactacaaatggagcccaagatgcagtccaagactaagatctaccgcagacccctggaccggcctgttagcccacgatctgatgttaatgacatcaaaggcacccctcctgaggaaatctcagctgcacaacctctactacgccccaattcagcaggaagcagttagagcggtcgtcggccaacctccccaacagcacttaggttttcctgttgagatgggggactgagagacaggactagctggatttcctaggctgattaagaatccctaagcctagctgggaaggtgaccacatccacctttaaacacggggcttgcaacttagctcacacctgaccaatcagagagctcactaaaatgctaattaggcaaagacaggaggtaaagaaatagccaatcatttattgcctgagagcacagcaggagggacaatgatcgggatataaacccaagttttcgagccggcaacggcaaccccctttgggtcccctccctttgtatgggagctctgttttcatgctatttcactctattaaatcttgcaactgcaaaaaaaaaaaaaaaaaaaaaa

SEQ ID NO: 10 - DNA, 2372 bp, Human
  misc_feature (1191)..(1191): n = any nucleotide
actgcactcttctggtccatgtttcttacggctcgagctgagcttttgctcaccgtccaccactgctgtttgccaccaccgcagacctgccgctgactcccatccctctggatcctgcagggtgtccgctgtgctcctgatccagcgaggcgcccattgccgctcccaattgggctaaaggcttgccattgttcctgcacggctaagtgcctgggtttgttctaattgagctgaacactaatcactgggttccatggttctcttctgtgacccacggcttctaatagaactataacacttaccacatggcccaagattccattccttggaatccgtgaggccaagaactccaggtcagagaatacgaggcttgccaccatcttggaagcggcctgctaccgtcttggaagtggttcaccaccatcttgggagctctgtgagcaaggaccccccggtaacattttggcaaccaacgacggacatccaaagtgatgggaaacgttccccgcaagacaaaaacgcccctaagacgtattctggagaattgggaccaatttgaccctcagacactaagaaagaaacgacttatattcttctgcagtgccgcctggcactcctgagggaagtataaattataacaccatcttacagctagacctcttttgtagaaaaggcaaatggagtgaagtgccataagtacaaactttcttttcattaagagacaactcacaattatgtaaaaagtgtgatttatgccctacaggaagccttcagagtctacctccctatcccagcatccccgactccttccccaactaataaggaccccccttcaacccaaatggtccaaaaggagatagacaaaagggtaaacagtgaaccaaagagtgccaatattccccaattatgacccctccaagcagtgggaggaagagaattcggcccagccagagtgcatgtgcctttttctctcccagacttaaagcaaataaaaacagacttaggtaaattctcagataaccctgatggctatattgatgttttacaagggttaggacaattctttgatctgacatggagagatataatgtcactgctaaatcagacactaaccccaaatgagagaagtgccaccataactgcagcctgagggtttggcgtctctggtatctcagtcaggtcaatggatanggatgacaacagaaggaaaganaatgattccccacaggccagcaggcagttcccagtctagaccctcattgggacacagaatcagaacatggagattggtgctgcagacatttgctaacttgtgtgctagaaggactaaggaaaactaggaagaagtctatgaattactcaatgatgtccaccataacacagggaagggaagaaaatcctactgcctttctggagagactaagggaggcattgaggaagcgtgcctctctgtcacctgactcttctgaaggccaactaatcttaaagcgtaagtttatcactcagtcagctgcagacattagaaaaaacttcaaaagtctgccgtaggcccggagcaaaacttagaaaccctattgaacttggcaacctcggttttttataatagagatcaggaggagcaggcggaacaggacaaacgggattaaaaaaaaggccaccgctttagtcatgaccctcaggcaagtggactttggaggctctggaaaagggaaaagctgggcaaattgaatgcctaatagggcttgcttccagtgcggtctacaaggacactttaaaaaagattgtccaagtagaagtaagccgccccttcgtccatgccccttatttcaagggaatcactggaaggcccactgccccaggggacaaaggtcttttgagtcagaagccactaaccagatgatccagcagcaggactgagggtgcctggggcaagcgccatcccatgccatcaccctcacagagccctgggtatgcttgaccattgagggccaggaaggttgtctcctggacactggtgcggtcttcttagtcttactcttctgtcccggacaactgtcctccagatctgtcactattctgagggggtccntaagacgggcagtcactagatactttttcccagccactaagttatgaactggggagctttattcttttcacatgcttttctaattatgcttgaaagccccactaccttgttagggagagacattctagcaaaagcaggggccattatacacctgaacataggagaaggaacacccgtttgttgtncccctgcttgaggaaggaattaatcctgaagtctgggcaacagaaggacaatatggacgagccaaagaatgcccgtcctgttcaagttaaactaaagg

SEQ ID NO: 11 - DNA, 7582 bp, Human
  misc_feature (198)..(198): n = any nucleotide
caacaatcgggatataaacccaggcattcgagctggcaacagcagcccccctttgggtcccttccctttgtatgggagctgttttcatgctatttcactctattaaatcttgcaactgcactcttctggtccatgtttcttacggctcgagctgagcttttgctcaccgtccaccactgctgtttgccaccaccgcanacctgccgctgactcccatccctctggatcctgcagggtgtccgctgtgctcctgatccagcgargcgcccattgccgctcccaattgggctaaaggcttgccattgtncctgcacggctaagtgcctgggtttgttctaattgagctgaacactantcactgggttccatggttctcttctgtgacccacggcttctaatakaactataacacttaccacatggcccaagattccattccttggaatccgtgaggscaacgaactccaggtcagagaatacgargcttgccaccatcttggaagcggcctgctaccrtcttggaagtggttcaccaccatcttgggagctctgtgagcaaggaccccccggtracattttggcraccamsracggacatccmaagtgatgggaaacgttccccgcaagacaaaaacgcccctaagacgtattctggaraattgggamcaatttgaccctcagacactaagaaagaaacgacttatattcttctgcagtgccgcctggcactcctgagggaagtataaattataacaccatcttacagctagacytcttttgtagaaaaggcaaatggagtgaagtgccataagtacaaactttcttttcattaagagacaactcacaattatgtaaaaagtgtgatttatgccctacaggaagccttcagagtctacctccctatcccagcatccccgactccttccccamytaataaggaccccccttcaacccaaatggtccaaaaggagatagacaaaagggtaaacagtgaaccaaagagtgccaatattccccaattatgacccctcccaagcagtgggaggaagagaattcggcccagccagagtgcatgtgcyttttyytctcccagacttaaagcaaataaaaacagacttaggtaaattctcagataaycctgatggctatattgrtgttttacaagggttaggacaattctttgatctgacatggagagatatatatgtcactgctaaatcagacactaaccccaaatgagagaagtgccaccataactgcagcctgagrgtttggcgatctctggtatctcagtcaggtcaatggatanggatgacaacagaaggaaaganaatgattccccacaggccagcargcagttcccagtctasaccctcattggggacacagaaatcagtaacatgggagattggtgctgcagacatttgctaacttgtgtgctasaaggactaaggaaaactasgaagaaartctaygaattactcaatgatgtccaccataacacaggggaagggaagaaaatcctactgcctttctggagagactaagggaggcattgaggaagcgtgcctctctgtcacctgactcttctgaaggccaactaatcttaaagcgtaagtttatcactcagtcagctgcagacattagaaaaaacttcaaaagtctgccgtaggcccggagcaaaacttagaaaccctattgaacttggcaacytcggttttttataatagagatcaggaggagcaggcggaacaggacaaacgggattaaaaaaaaggccaccgctttagtcatgaccctcaggcaagtggactttggaggctctggaaaagggaaaagctgggcaaattgaatgcctaatagggcttgcttccagtgcggtctacaaggacactttaaaaaagattgtccaagtagaagtaagccgccccttcgtccatgccccttatttcaagggaatcactggaaggcccactgccccaggggacaaaggtcttttgagtcagaagccactaaccagatgatccagcagcaggactgagggtgcctggggcaagcgccatcccatgccatcaccctcacagagccctgggtatgcttgaccattgagggccaggaaggttgtctcctggacactggtgcggtcttcttagtcttactcttctgtcccggacaactgtcctccagatctgtcactattctgagggggtccntaagacgggcagtcactagatactttytcccagccactaagttatgaactggggagctttattcttttcacatgcttttctaattatgcttgaaagccccactaccttgttagggagagacattctagcaaaagcaggggccattatacacctgaacataggagaaggaacacccgtttgttgtncccctgcttgaggaaggaattaatcctgaagtctgggcaacagaaggacaatatggacgagccaaagaatgcccgtcctgttcaagttaaactaaaggattccacttcctttccctaccaaaggcagtaccccctcagacccaaggcccaacaaggattccaaaagattgttaaggacttaaaagcccaaggcttagtaaaaccatgcataactccctgcagtaattccgtagtggattgaggaggcacagaaacccagtggacagtggagggttagtgcaagatctcaggattatcaatggaggccgttgtccttttatacccagctgtacctagcccttatactgtgmyttcccaaataccagaggaagcagagtggtttacastcctggaccttmaggatgccttcttctgcatccctgtacatcctgactctcaattcttgtttgcctttgaagatacttcaaacccarcatctcaactcacctggactrttttaccccaagggttcagggatagyccccatctatttggccaggcattagcccaagacttgagycartymtcatacctggacactcttgtccttcrgtakgtggatgatttacttttrgcygccyrttcagaaaccttgtgccatcaagccacccaagcrctcttmaatttcctcgcyacctgtggctacawggtttccaaacsarargctcarctctgctcacagcaggttaaatacttaggrctaarattatccaaaggcaccarggccctcagtgaggaayryatccagcctatactggcttatcctcatcycaaaaccctaaagcaactaagrgrrttccttggcrtaayaggyttctgccgaawatggattccccaggtwtggcraaatagccaggycattawatacastaattaaggaaactcagaaagccaatacccatttartaagatggayamctgaagymraagtggctttccaggcccctaaagaaggccttaaacccaagycccagtgttaagyttgccaacrgggcaagacttttsttyatayrtcacagaaaaaaacagraayagctctrggagtccttacacagrtccragggaygagcttgcaaccyrtggcryacctgastaaggaaaytgatgtagtggcaaagggttgrcytcattgtttaygggtagtggtggcagtagcagtyktagtatctgaagcagttaaaataatacagggragagatcttactgtgtggacatctcatgakgtgaayrgcatactcactgctaaaggagacttgtggctgtcagacaacygtttacttaaatrtcaggctctattacttgaarggccagtgctgcractgtgcacttgtgcaactcttaacccagycncatttcttccagacaatgaagaaaagataraayataactgtcaacaartaatttctcaaacctatgccactcgaggggaccttytagargttccyttgactgatccygaccttcaacttgtatactgatggaagttcctttgtagaaaaaggacttcgaaaagyggggtatgcagtggtcagtgataatggaatayttgaaagtaatcccctcactccaggaactagtgctyagctrgcagaactaatagccytcaytkgggcactagaattaggagaagraaaaagggyaaatatatatacagactctrartatgctyacctagtcntccatgcccatgmrgcaatatgsaragaaagggaattcctaacttcygagrgaacacctatcamacatcaggaagccattaggarattattaytggcwgtacagaaacctaragaggtggmagtcttacactgcyggggtcatcanaaaggaaagraaagggaaatasaagrgaaytgccaagcakatattgaagcmaaaagagctgcaaggcaggaccctccattagaaatgcttattaaacttcccttagtatagggtaatcccttccgggaaaccaagccccagtactcagcaggagaaacagaatggggaacctcacgaggcagttttctcccctcgggacggttagccactgaagaagggaaaatacttttgcctgcaactatccaatggaaattacttaaaacccttcatcaaacctttcacttaggcatcgatagcacccatcaratggccaaatcattatttactggaccaggccttttcaaaactatcaagcarataktcagggcctgtgaaktgtgccararaaataatcccctgcctyatcgccaagctccttcaggaraacaaaraacaggccattaccctgraraaractggcaactgattttacccacaagcccaaacctcagggatttcagtatctactagtctgggtaratactttcacgggttgggcaraggccttcccctgtaggacagaaaaggcccaagaggtaataaaggcactagttcatgaaataattcccagattcggacttccccgaggcttacagagtgacaatagccctgctttccaggccacagtaacccagggagtatcccaggcgttaggtatacgatatcacttacactgcgcctgaaggccacagtcctcagggaaggtcgagaaaatgaatgaaayactcaaaggacatctaaaaaagcaaacccaggaaacccacctcacatggcctgytctgttgcctatagccttaaaaagaatctgcaactttccccaaaaagcaggacttagcccatacgaaatgctgtatggaaggcccttcataaccaatgaccttgtgcttgacccaagacagccaacttagttgcagacatcacctccttagccaaatatcaacaagttcttaaaacattacaaggaacctatccctgagaagagggaaaagaactattccacccwwgtgacatggtattagtcaagtcccttcyctctaattccccatccctagatacatcctgggaaggaccctacccagtcattttatytaccccaactgcggttaaagtggctggagtggagtcttggatacatcacacttgagtcaaatcctggatactgccaaaggaacctgaaaatccaggagacaacgctagctattcctgtgaacctctagaggatttgcgcctgctcttcaaacaacaaccaggaggaaagtaactaaaatcataaatcccccatggscctcccttatcatatttttctctktastgttstttyaccctstttcactctcactgcaccccctccatgccgctgtatgaccagtagctcccctyaccmagagtttctatggagaatgcagcgtcccggaaatattgatgccccatcgtataggagtctttstaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcaccsgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcargccatatgtttcaatccctgtacctgaacaatggaacaacttcagcacagaaataaacaccacttccgttttagtaggacctcttgtttccaatstggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgcccccyatgrccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcraaatcgaagagctttagactygctaaccgctgaragagggggaacctgtttatttttaggggaagaatgctgttattatgttaatcaatccggaatcgtcactgagaaagttraagaaattcsagatcgaatacaacgtakagcagargagcttcgaaacactggaccctggggcctcctcagccratggatgccctggattctccccttcttaggacctctagcagctataatattgctactcctctttggaccctgtatctttracctccttgttaactttgtctcttccagaatcgaagctgtraaactacaaatggagcccaagatgcagtccaagactaagatctaccgcagacccctggaccggcctgytagcccacgatctgatgttaatgacatcaaaggcacccctcctgaggaaatctcagctgcacaacctctactacgccccaattcagcaggaagcagttagagcggtsgtcggccaacctccccaacagcacttaggttttcctgttgagatgggggactgagagacaggactagctggatttcctaggctgaytaagaatccytaagcctagstgggaaggtgaccacatccacctttaaacacggggcttgcaacttagytcacacctgaccaatcagagagctcactaaaatgctaattaggcaaagacaggaggtaaagaaatagccaatcatytattgcmtgagagcacagcaggagggacaatgatcgggatataaacccaagtyttcgagccggcaacggcaaccccctttgggtcccctccctttgtatgggagctctgttttcatgctatttcactctattaaatcttgcarctgcr

SEQ ID NO: 12 - DNA, 2563 bp, Human
actgcactcttctggtccatgtttgttacggctcgagctgagcttttgctcgccatccaccactgctgtttgccaccgttgcagacccactgctgacttccatccctctggatctggcagggtgtctgctgtgctcctgatccagcgaggggcccattgccactcccaatcgggctaaaggcttgccattgttcctgcatggctaagtgcccaggttcatcctaattgagctgaacactagtcactgggttccacagttctcttccatgaaccacggcttttaatagagctataacactcatcgcaaggcccaagattccattccttggaatctgtgaggccaagaaccctaggtcagagaacacgaggcttgccaccatcttggaagcagcctgccaccatctgggaagcggcctgccaccatcttggaagccgcccgccaccatcttgggagctctgggagcaaggacctccccgcaacccagtaacatttagcgaccacgaagggacctccaaagcggtaatattggaccactttcacttgctattctgtcctatccttccttagaattggaggaaaataccggacacctgtcggccggttaaaaacgattagcgtggcctccggacttaagaatcaggtgtgaggctatctggggaagggctttctaacaacccccaaccrttctgggttgggaatgttggtctgcctggagccagcttccactttcaattttcctggggaagccaagggccgactagaggcagaaagctgttgtcccaaattcccggcagtagccggttgagatcatggcgcagccagaagtctttactccacagtcacccatgcatgcgcccctatctttccttctgacccatacctcctgggtcctaaccatgactttcttaaaagggtagccccaaaattctccttacctctgaatctacttcctctgatccctgcctcctaggtgctaatggttcagactttcatttcctctagcaagttgtatytccaaagggatataaggaagctctacactgtatccttaggcatctaggctctaaacccagggagtcttgtccctgatgtcccaaccgatttaggtatatagttctcgacatgggcagttatgtgggacccattccccaccacccttgccagggccccaagtttgtaaatggctaagagaggaaagtgagagagagagagacagagtgagacacagagagagggagagacagagagagagacagagaggagagagacacagagaggggagagacacagagaggagaagggggcagagagaccaagagggagtcymagagagagagaaagaagaagaaatagtagaaaaaaaagtgtgccctattcctttaaaagccagggtaaatttaaaaaacctatacttgataattgaaggtcttctccatgaccctgtaacactctaatactaccttgttctcagtgtaaacaagggtgttagcctgaaaacactgagaccgctgacacccatagctttcctataaaaaatccttaacccagtaacccgcagatggcccgcatgcattcaatctgtagtggcaactgctttgctaacaagaataaagtggaaaagtaacttttagaggaaacctcattgtgagcacacctcaccagttcagaattattctaagtcaaaaaagcaaaaaggtagcttactaactcaaaaatcttaaagtatggggttattttgttagaaaaaggtaatttaacactaatcactgataattcccttaacccagaagatttcctaacaggagatttaaatcttaattaccatacaaaggtctgaccagacctaggaggaactcccttcagtacaggatgatagatggttcctcccaggtgaatgaaaaaaaaatcacaatgggtattcagtaattgatagggagactcttgtggaagcagagttagaaaaactgcctaataattggtctccccaaacctgcgagctgtttgcactcagccaagccttaaagtacttctagaatcaaaaagattatctcaatcctgactcaaaaggttacctacaccctctgtgaaacgaatttacttaagaactgtttatgggactgcatcttgatggggcagctgggttgtcatgaaatactcaggaatgcagcctagctctaggactcacccctgagcacaaaggcaatgttgggcatgctggtaaaggaccactagaatccagcagtccgaaccctttctttgggttaagaaaggcgggaaaacaggcgcaggactgctacattggtaagcgtaactaatccaataagcagaggtccatgggtggtgacacactctggaaaggaataagcattagraccatagaggacgctctacgactaatgctcgtcggaaaatgactagaggtgctggcatccctatgttcttttttcagatgggaaatgttccccctcaaggcaaaaacacccctaagatgtattctggacaattgggaccaatttgaccctcagactctaagaaagaaacgacttatattcttctgcagtg

SEQ ID NO: 13 - DNA, 2585 bp, Human
  misc_feature (726)..(726): n = any nucleotide
tcagggatagcccccatctatttggccaggtattagcccaagacttgagccagttctcatacttggacactcttgtcctttggtatgtggatgatctacttttagccacctgttcagaaaccttgtgccatcaagccaaccaagtgctcttaaacttcctcgccacctgtggctacaaggtttccaaaccagaggctcagctctgcttacagcaggttaaatacttagggctaaaattatccaaaggcaccagggccctcagtgaggaacgtatccagcctatactggcttatcctcatcccaaaaccctgaagcaattaagagggttccttggcataaaaggctgctgttgaatatggattcccaggtacaatgaaatagccaggccattatacacactaattacgggaactcagaaagccaatacccatttagtagaatggacacctgaagcagaagcggctttccaggccctaaagaaggccctaatccaagccccagtgttaagcttgccaatggagcaagacttttctttatatgtcacagaaaaaaaaacaggaatagctctagaagtccttacacaggtccgagggaccagcttacaacacatggcatacctgagtaaggaaactgatgtagtggcaaagggttggactcattgtttacaggtagtggcagcagtagcagtcttagcatctgaagcagttaaaatgatacagggaaganatcttactgtgtggacatctcatgatgtgaacggcatactcactgctaaaggagactgtggctgtcagacaaccatttgcttaaatatcaggctctatcacttgaanggccagtgctgccactgtgcacttgtgcaactcttaacccacccacatttcttccagacaatgaagaaaagatagaacataactgtcaacaagtgattgttcaaacctacaccgctcgaagggaccttctagaggttcccttgactgatcctgagctcaacttctatactgatggaagttccttttgtagaaaaaggacttcgaaaggcgggtatgcagtggccagtgataatggaatacttgaaagtaatcccttcactccagaaactagcattcagctggcagaattaatagccttcacttgggcattagaacacaggagaaggaaaaggagtaaatatatatacagactccaagtatgcttacttagtcctccatgcccatgcagcaatatagagagaaagcgaattcctaacttctgagggaacacctatcaaacatcaggaagccattaggagattattactggctgtacagaaacctagaggtggcagtcttacatggccgagatcatcagaaaggaaaagaaagggaaatagaagggaactgccaagtggatattgaagccaaaagagctgcaaggcgggaccctccattagaaatgcttatagaaggacccctagtacagggcaatccccttcaggaaaccaagccccaatactcagcagaagaaatggaatggggaacctcatgaggacatagtttcctcccctcaggatggctagccaccaaagaaggaaaaatacttttgcctgcagctaaccaatggaaattacttaaaacccttcaccaaacctttcgcttaggcattgatagcacccatcagatggctaaatcattatttactagaccacaccttttcaaaactatcaagcagacagttagggcctgtgaagtgtgccaaagaaataatcccctgccttatcgccaaactccttcaggagaaaaaagaacaggccattacccaggagaagagtggcaactagattttacccacatgcccaaatctcagggatttcagtatctactagtctgggtagatactttcactggttgggcggaggccttcccttgtaggacagaacaggcccatgaggtaataaaggcactaattcatgaaataattcccagatttggatttccccaaggcttacagagtgataacggccccactttcaaggctacagtaacccagggagtatcccagacattagacatacaatatcacttacactgagcccggaggccacaatcctcaggaaagttgagaaaatgaatgaaacgctcaaatgacatctaaaaaagctaacctaagaaacccacctctcatggtttgctctgttgcctatagccttagtaagaatccgaaactctccccaaaaagcgggactcagcccatacgaaatgctgtatggacggcccttcctaaccaatgaccttgtgcttgacctagagatggccaacttagttgcagatatccctccttagccaaatatcaacaagttcttaaaacgtcacagggaacctgtccctgagaggagggaaaggaattattccaacctggtgacatggtattagtgaagtcccttccctccaactccccatcccctggatacatcctgggaaggaccctactcagtcattttatctatcccaaccgcggttaaaatggctggagtagaatcttggatacatcacattcgagtcaaaccctagatactgccacaaggaacctgaaaatccaggagacaa

SEQ ID NO: 14 - DNA, 2575 bp, Human
gggatagcccccatctatttggccaggcattagcccaagacttgaagccaattctcatacctggacactcttctcctttggtatgtggatgatttacttttagcttcctgttcagaaaccttgtgccatcaagccacccaagcactcttaaatttcctcgctacctgtggctacaaggtttccaaaccaaagacccagctctgctcacagcaggttaaatacttggggctaaaattatccaaaggcaccagggccctcagtgaggaacgtatcaagcctatactggcttatcctcatccccaaatcctaaagcaactaagagagttccttagcataacaggtttctgctgaatatggattcccaggtatggcaaaatagccagaccattatatacgctaattaaggaaactcagaaagccaatacccatttagtaagatggatacctgaagcagaagcagctttccaggccctaaagagggccctaacccaagccccagtgttaagcttgccaacagggcaagactttacttcgtatgtcacagaaaaaacaggaaatagctctaggagtccttacacaagtctgagggatgagcttgcaacccatggcatacctgagtaaggaaattgatgtagtggcaaagggttggcctcattgtttatgggtagtggcggcagtagcagtcttagcatctgaagcagttaaaatgatacagggaagagatcttactgtgtggacatctcatgatgtgaatggcatactcactgctaaaggagacttgtggctgtcagacaaccatttacttaaatatcaggctgtattacttgaagggccagtgcagcaactgcgcagttgtgcagctcttaacccagccacatttcttccagacaatgaagatagaacataactgccaacaagtaatttctcaaacctaggccgctcgagggaaccttttagaggttcccttaactgatcccgacctcaacttgtatactgatggaagttcctttgtagaaaaaggactttgaaaagtggggtatgcagtgctcagtgataatggaatacttgaaaataatcccttcattccaggaaccagcgttcagctggcagaattaatagccctcactcgggcattagaattaggagaaggaaaaagggtaaatacacatacagattctaagtatgtttacttagtcctccgtgcccacgcagcaatatggagagaaagggaatgcttaacttctgagggaacacctatcaaacatcaggaagttattaggagattattattggctatacagaaacctaaagaggtggcagtcttacactgctggggtggtcagaaagaaaaggaaagggaaataaaagggaactgccaagcggatattgaagccaaaagagccgcaaggcaggaccctccattagaaatgcttatagaaggacccctagtatggggtaatcccctccgggaaaccaagccccaatacttagaaaaagaaatagaatggggaacctcacgaggacatagtttcctcccctcaggatggctagccaccgaagaaggaaaaatacttttgcctgcagctaaccaatggaaattacttaaaacccttcaccaaacctttcacttagacattgatagcacccatcagatggccaaatcattatttactggaccaggccttttcaaaactatcaagcagctagtcagggcctgtgaagtgtgccgaagaaataatcccatgccttatcaccaagctccttcaggagaacaaagaacaggccattacccaggagaagrvtggcaactagattttacccacatgcccaaatctcagggatttcagtatctactagtttgggtagatactttcactggttgggcagagaccttcccctgtaagacagaaaagtcccaagaggtaataaaggcattagttcatgaaataattcccagattcagacttccctgaggcttacagagtgacaatggccctgctttcaaggctacagtaacccaggagtatcccaggtgttaggtatacaatatcacttacactgcgcctggaggcagtcctcagggaaggccgagaaactgaatgaaacactcaaacgacatctaaaaaaagctaacccaggaaaaccacctcacatggcctgctctgttgcctatagccttactaagaatccaaaactctccccaaaaagcaggacttagcccatacgaaatgctatatggatagcccttcctaaccaatgaccttgtgcttgactgagagagagccaacttagttgcagacatcacctccttatccaaatatcaacaagttcttaaaacattacaaggagcctgtccccgagaagaggggaaggaactattccaccctggtgacatggtattagtcaagtcccttccctctaattctcattgcctagatatatcctgggaaggaccctacccagtcattttatctaccccaaccgcagtaaaagtggctggagtggagtcttggatacatcacactcgagtcaaaccctggatattaccaaaggaacctgaaaatccaggagacaa

SEQ ID NO: 15 - DNA, 783 bp, Artificial Sequence
  Consensus Sequence
tgagagacaggactagctggatttcctaggcygactaagaatccytaagcctagstgggaaggtgaccacrtccacctttaaacacggggcttgcaacttagytcacacctgaccaatcagagagctcactaaaatgctaattaggcaaagacaggaggtaaagaaatagccaatcatytattgcmtgagagcacagcaggagggacaayratcgggatataaacccargyhttcgagcyggcaacrgcagmccccctttgggtcccytccctttgtatgggagctctgttttcatgctatttcactctattaaatcttgcarctgcrctcttctggtccatgtttcttacggctygagctgagctttygctcrccrtccaccactgctgtttgccrccaccgcanaccygccgctgactcccatccctctggatcmtgcagggtgtccgctgtgctcctgatccagcgargcrcccattgccgctcccaatygggctaaaggcttgccattgtncctgcayggctaagtgcctgggttyrtyctaattgagctgaacactantcactgggttccatggttctcttctgtgacccacrgcttctaatagarctataacactyaccrcatggcccaagrttccattccttggaatccrtrargscaacgaacyccasgtcagagaayacgargcttgccaccatcttggaagcggcctgctaccatcttggaagtggttcaccaccatcttgggagctctgtgagcaaggacccccmrgtraca

SEQ ID NO: 16 - DNA, 20 bp, Artificial Sequence
  PCR primer or probe
tgtccgctgtgctcctgatc

SEQ ID NO: 17 - DNA, 21 bp, Artificial Sequence
  PCR primer or probe
atgcactctggctgggccaat

SEQ ID NO: 18 - DNA, 21 bp, Artificial Sequence
  PCR primer or probe
accatttgaccctcagacact

SEQ ID NO: 19 - DNA, 24 bp, Artificial Sequence
  PCR primer or probe
aaccctttgccactacatcaattt

SEQ ID NO: 20 - DNA, 21 bp, Artificial Sequence
  PCR primer or probe
tcagggatagcccccatctat

SEQ ID NO: 21 - DNA, 22 bp, Artificial Sequence
  PCR primer or probe
ttgtctcctggattttcaggtt

SEQ ID NO: 22 - DNA, 20 bp, Artificial Sequence
  PCR primer or probe
ggaccctacccagtcatttt

SEQ ID NO: 23 - DNA, 20 bp, Artificial Sequence
  PCR primer or probe
atcaggagcacagcggacac

SEQ ID NO: 24 - DNA, 22 bp, Artificial Sequence
  Probe or primer
ggacatccaaagtgatacatcc

SEQ ID NO: 25 - DNA, 21 bp, Artificial Sequence
  Probe or primer
aatgtatggcctgaagtgcag

SEQ ID NO: 26 - DNA, 22 bp, Artificial Sequence
  Probe or primer
cttcccaggatgtatcactttg

SEQ ID NO: 27 - DNA, 24 bp, Artificial Sequence
  Probe or primer
cactgcagaagaatataagtcgtt

SEQ ID NO: 28 - DNA, 21 bp, Artificial Sequence
  Probe or primer
gcttccaagatggtggcaagc

SEQ ID NO: 29 - DNA, 678 bp, Artificial Sequence
  Ppol-MSRV probe
tcagggatagcccccatctatttggccaggcattagcccaagacttgagccagttctcatacctggatattcttgtcctttggtatgcggatgatttacttttagccgcccgttcagaaaccttgtgccatcaagccacccaagtgctcttaaatttcctcgccacctgtggctacaaggtttccaaaccaaaggctcagctctgctcacagcagaaggctatttaccctaaatacttagggctgaaattatccaaaggcaccagggccctcagtgaggaatgtatccagcctatactggcttatccttatcccaaaaccctaaaacaactaagaaggttccttggcataataggcataacaggcataacaggtttctgctgaatatggattcccaagtacggcaaaatagccagaccattatatacactaattaaggaaactcagaaagccaatacccatttagtaagatggacacctgaagcagaggcagctttccaggccgtaaagaacaccctaacccaagccccagtgttaagcttgccagcggggcaagacttttctttctgtgtcacagaaaaaataggaatagctntaggagtccttacacaggtccgagggaccagcttgcaacccatggcatacctgagtaaggaaattgatgtagtggcaaagggtt

SEQ ID NO: 30 - DNA, 536 bp, Artificial Sequence
  Pgag-LB19 probe
ccaatctccatgttgtatccccttccccaactaataaggacccccctttcaacccaaacagtccaaaaggacatagacaaaggagtaaacaatgaaccaaagagtgccaatattccctggttatgcaccctccaagcggtgggagaagaattcggcccagccagagtgcatgtacctttttctctctcacacttgaagcaaattaaaatagacctaggtaaattctcagatagccctgatggctatattgatgttttacaaggattaggacaatcctttgatctgacatggagagatataatattactgctaaatcagacgctaacctcaaatgagagaagtgctgccataactggagcccgagagtttggcaatctctggtatctcagtcaggtcaatgataggatgacaacggaggaaagagaacgattccccacagggcagcaggcagttcccagtgtagctcctcattgggacacagaatcagaacatggagattggtgccgcagacatttaaagctttccccgggtaccga

SEQ ID NO: 31 - DNA, 591 bp, Artificial Sequence
  Penv-C15 probe
ccatggccatctacactgaacaagatttatacaatcatgtcgtacctaagccccacaacaaaagagtacccattcttccttttgttatcagagcaggagtgctaggcagactaggtactggcattggcagtatcacaacctctactcagttctactacaaactatctcaagaaataaatggtgacatggaacaggtcactgactccctggtcaccttgcaagatcaacttaactccctagcagcagtagtccttcaaaatcgaagagctttagacttgctaaccgccaaaagagggggaacctgtttatttttaggagaagaacgctgttattatgttaatcaatccagaattgtcactgagaaagttaaagaaattcgagatcgaatacaatgtagagcagaggagcttcaaaacaccgaacgctggggcctcctcagccaatggatgccctgggttctccccttcttaggacctctagcagctctaatattgttactcctctttggaccctgtatctttaacctccttgttaagtttgtctcttccagaattgaagctgtaaagctacagatggtcttacaaatctaga

SEQ ID NO: 32 - DNA, 364 bp, Artificial Sequence
  Ppro-E probe
ctaacctgaggatccagcagcaggactgagggtgcccggggcaagtgccagcccatgccatcaccctcagagccccgggtatgtttgaccattgagagccaggaagttaactgtctcctggacactggcgcagccttctcagtcttactttcctgtcccagacaattgtcctccagatctgtcactatccgaggggtcctaggacagccagtcactacatacttctctcagccactaagttgtgactggggaactttactcttttcacatgcttttctaattatgcctgaaagccccactcccttgttagggagagacattttagcaaaagcaggggccattatacacctgaacaagcttgaaa

SEQ ID NO: 33 - protein, 538 aa, Human
MGLPYHIFLCSVLSPCFTLTAPPPCRCMTSSSPHPEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTGVHGTSSPYKGLDLSKLHETLRTHTRLVSLFNTTLTGLHEVSAQNPTNCWICLPLNFRPYVSIPVPEQWNNFSTEINTTSVLVGPLVSNVEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLRNRRALDLLTAERGGTCLFLGEECCYYVNQSGIVTEKVEEIPDRIQRIAEELRNTGPWGLLSRWMPWILPFLGPLAAIILLLLFGPCIFDLLVNFVSSRIEAVKLQMEPKMQSKTKIYRRPLDRPASPRSDVNDIKGTPPEEISAAQPLLRPNSAGSS

SEQ ID NO: 34 - protein, 52 aa, Human
MEPKMQSKTKIYRRPLDRPASPRSDVNDIKGTPPEEISAAQPLLRPNSAGSS

SEQ ID NO: 35 - protein, 48 aa, Human
MLMTSKAPLLRKSQLHNLYYAPIQQEAVRAVVGQPPQQHLGFPVEMGD

SEQ ID NO: 36 - DNA, 20 bp, Human
atccaaagtggtgagtaata

SEQ ID NO: 37 - DNA, 20 bp, Human
cttttttcagatgggaaacg

SEQ ID NO: 38 - DNA, 10 bp, Human
atccmaagtg

SEQ ID NO: 39 - DNA, 20 bp, Human
caggaggaaagtaactaaaa

SEQ ID NO: 40 - DNA, 10 bp, Human
atgggaaacg

SEQ ID NO: 41 - DNA, 20 bp, Human
ccatccctagatacatcctg

SEQ ID NO: 42 - DNA, 20 bp, Human
tctcttccagaatcgaagct

SEQ ID NO: 43 - DNA, 873 bp, Human
ccctggggcgggcttcctttctgggatgagggcaaaacgcctggagatacagcaattatcttgcaactgagagacaggactagctggatttcctaggccgactaagaatccctaagcctagctgggaaggtgaccacgtccacctttaaacacggggcttgcaacttagctcacacctgaccaatcagagagctcactaaaatgctaattaggcaaagacaggaggtaaagaaatagccaatcatctattgcctgagagcacagcaggagggacaacaatcgggatataaacccaggcattcgagctggcaacagcagcccccctttgggtcccttccctttgtatgggagctgttttcatgctatttcactctattaaatcttgcaactgcactcttctggtccatgtttcttacggctcgagctgagcttttgctcaccgtccaccactgctgtttgccaccaccgcagacctgccgctgactcccatccctctggatcctgcagggtgtccgctgtgctcctgatccagcgaggcgcccattgccgctcccaattgggctaaaggcttgccattgttcctgcacggctaagtgcctgggtttgttctaattgagctgaacactagtcactgggttccatggttctcttctgtgacccacggcttctaatagaactataacacttaccacatggcccaagattccattccttggaatccgtgaggccaagaactccaggtcagagaatacgaggcttgccaccatcttggaagcggcctgctaccatcttggaagtggttcaccaccatcttgggagctctgtgagcaaggaccccccggtaacattttggcaaccacgaacggacatcca

SEQ ID NO: 44 - DNA, 815 bp, Human
tcgtcggccaacctccccaacagcacttaggttttcctgttgagatgggggactgagagacaggactagctggatttcctaggctgactaagaatccctaagcctagctgggaaggtgaccacatccacctttaaacacggggcttgcaacttagctcacacctgaccaatcagagagctcactaaaatgctaattaggcaaagacaggaggtaaagaaatagccaatcatctattgcctgagagcacagcaggagggacaatgatcgggatataaacccaagtcttcgagccggcaacggcaaccccctttgggtcccctccctttgtatgggagctctgttttcatgctatttcactctattaaatcttgcaactgcactcttctggtccatgtttcttacggcttgagctgagctttcgctcgccatccaccactgctgtttgccgccaccgcagacccgccgctgactcccatccctctggatcatgcagggtgtccgctgtgctcctgatccagcgaggcacccattgccgctcccaatcgggctaaaggcttgccattgttcctgcatggctaagtgcctgggttcatcctaattgagctgaacactagtcactgggttccatggttctcttctgtgacccacagcttctaatagagctataacactcaccgcatggcccaaggttccattccttgaatccataaggccaagaaccccaggtcagagaacacgaggcttgccaccatcttgggagctctgtgagcaaggacccccaagtaacacaaccatgagggtgcaaatgcatggg

SEQ ID NO: 45 - DNA, 425 bp, Human
caattcagcaggaagcagttagagcggtggtcggccaacctccccaacagcacttaggttttcctgttgagatgggggactgagagacaggactagctggatttcctaggctgactaagaatccttaagcctaggtgggaaggtgaccacatccacctttaaacacggggcttgcaacttagctcacacctgaccaatcagagagctcactaaaatgctaattaggcaaagacaggaggtaaagaaatagccaatcatttattgcctgagagcacagcaggagggacaatgatcgggatataaacccaagttttcgagccggcaacggcaaccccctttgggtcccctccctttgtatgggagctctgttttcatgctatttcactctattaaatcttgcaactgcaaaaaaaaaaaaaaaaaaa

SEQ ID NO: 46 - DNA, 427 bp, Human
caattcagcaggaagcagttagagcggtcgtcggccaacctccccaacagcacttaggttttcctgttgagatgggggactgagagacaggactagctggatttcctaggctgactaagaatccctaagcctagctgggaaggtgaccacatccacctttaaacacggggcttgcaacttagttcacacctgaccaatcagagagctcactaaaatgctaattaggcaaagacaggaggtaaagaaatagccaatcatctattgcatgagagcacagcaggagggacaatgatcgggatataaacccaagtcttcgagccggcaacggcaaccccctttgggtcccctccctttgtatgggagctctgttttcatgctatttcactctattaaatcttgcagctgcgaaaaaaaaaaaaaaaaaaaa

SEQ ID NO: 47 - DNA, 600 bp, Human
caacaatcgggatataaacccaggcattcgagctggcaacagcagcccccctttgggtcccttccctttgtatgggagctgttttcatgctatttcactctattaaatcttgcaactgcactcttctggtccatgtttcttacggctcgagctgagcttttgctcaccgtccaccactgctgtttgccaccaccgcagacctgccgctgactcccatccctctggatcctgcagggtgtccgctgtgctcctgatccagcgaagcgcccattgccgctcccaattgggctaaaggcttgccattgttcctgcacggctaagtgcctgggtttgttctaattgagctgaacactagtcactgggttccatggttctcttctgtgacccacggcttctaatagaactataacacttaccacatggcccaagattccattccttggaatccgtgaggccaagaactccaggtcagagaatacgaagcttgccaccatcttggaagcggcctgctaccatcttggaagtggttcaccaccatcttgggagctctgtgagcaaggaccccccggtaacattttggcaaccacgaacggacatcca

SEQ ID NO: 48 - DNA, 530 bp, Human
atgggagctgttttcatgctatttcactctattaaatcttgcaactgcactcttctggtccatgtttcttacggctcgagctgagcttttgctcaccgtccaccactgctgtttgccaccaccgcagacctgccgctgactcccatccctctggatcctgcagggtgtccgctgtgctcctgatccagcgaagcgcccattgccgctcccaattgggctaaaggcttgccattgttcctgcacggctaagtgcctgggtttgttctaattgagctgaacactagtcactgggttccatggttctcttctgtgacccacggcttctaatagaactataacacttaccacatggcccaagattccattccttggaatccgtgaggccaacgaactccaggtcagagaatacgaagcttgccaccatcttggaagcggcctgctaccatcttggaagtggttcaccaccatcttgggagctctgtgagcaaggaccccccggtgacattttggcgaccaccaacggacatccc

SEQ ID NO: 49 - DNA, 486 bp, Human
  misc_feature (84)..(84): n = any nucleotide
actgcactcttctggtccatgtttcttacggctcgagctgagcttttgctcaccgtccaccactgctgtttgccaccaccgcanacctgccgctgactcccatccctctggatcctgcagggtgtccgctgtgctcctgatccagcgaggcgcccattgccgctcccaattgggctaaaggcttgccattgtncctgcacggctaagtgcctgggtttgttctaattgagctgaacactantcactgggttccatggttctcttctgtgacccacggcttctaatagaactataacacttaccacatggcccaagattccattccttggaatccgtgagggcaagaactccaggtcagagaatacgaggcttgccaccatcttggaagcggcctgctaccatcttggaagtggttcaccaccatcttgggagctctgtgagcaaggaccccccggtaacattttggcaaccacgaacggacatcca

SEQ ID NO: 50 - protein, 37 aa, Human
KIYRRPLDRPASPRSDVNDIKGTPPEEISAAQPLLRP

SEQ ID NO: 51 - protein, 35 aa, Human
MTSKAPLLRKSQLHNLYYAPIQQEAVRAVVGQPPQ

SEQ ID NO: 52 - protein, 33 aa, Rex PTLV-L
RLYNTLSLDSPPSPPKELPAPSRFSPPQPLLRP

SEQ ID NO: 53 - protein, 35 aa, Tat SIV-AGM
VTYHAPRTRRKKIRSLNLAPLQHQSISTKWGRDGQ

(19) United States
(12) Patent Application Publication
(10) Pub. No.: US 2004/0176314 A1
(43) Pub. Date: Sep. 9, 2004

(54) Endogenetic retroviral sequences, associated with autoimmune diseases or with pregnancy disorders

(75) Inventors: Frederic Beseme, Villefontaine (FR); Jean-Luc Blond, Lyon (FR); Olivier Bouton, Francheville (FR); Bernard Mandrand, Villeurbanne (FR); Francois Mallet, Villeurbanne (FR); Herve Perron, Lyon (FR)

Correspondence Address: OLIFF & BERRIDGE, PLC, P.O. BOX 19928, ALEXANDRIA, VA, 22320, US

(73) Assignee: BIO MERIEUX, Marcy L'Etoile (FR)

(21) Appl. No.: 10/717,580

(22) Filed: Nov. 21, 2003

Related U.S. Application Data
(63) Continuation of application No. 09/446,024, filed on Dec. 16, 1999, now abandoned.
Application No. 09/446,024 is a 371 of international application No. PCT/FR98/01442, filed on Jul. 6, 1998.

(30) Foreign Application Priority Data
Jul. 7, 1997 (FR) 97/08815

(51) Int. Cl. A61K 48/00; C07H 21/02

(52) U.S. Cl. 514/44; 536/23.1

(57) ABSTRACT

The invention concerns a genomic retroviral nucleic material, in an isolated or purified state, at least partially functional or non-functional, wherein the genome comprises a reference nucleotide sequence selected from the group including sequences of SEQ ID NOs: 1-15, their complementary sequences, and their equivalent sequences, in particular, nucleotide sequences having, for every series of 100 contiguous monomers, at least 70% and preferably at least 90% homology with the sequences of SEQ ID NOs: 1-15. The invention also concerns the application of this material.

The present invention relates to a new nucleic material of the endogenous retroviral genomic type, various nucleotide fragments comprising it or which are obtained from said material, as well as their use as marker for at least one autoimmune disease or a pathology which is associated with it, a pathological pregnancy or an unsuccessful pregnancy.

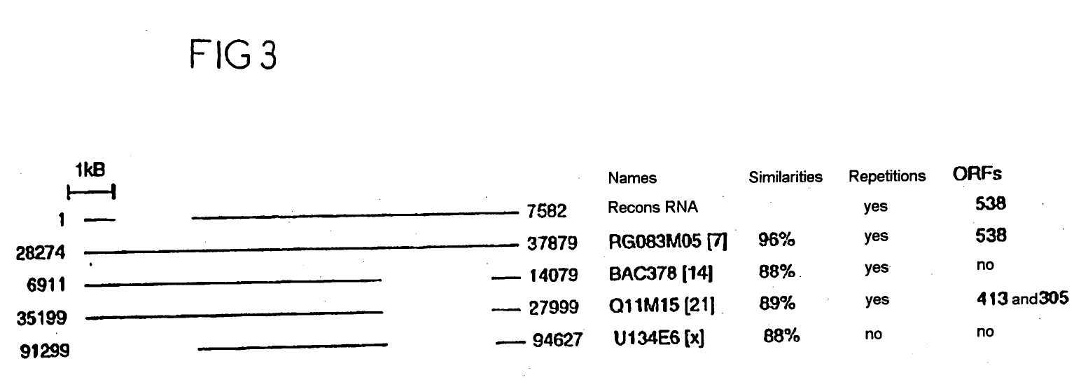

The screening of the cDNA library with the aid of the Ppol-MSRV probe (SEQ ID NO: 29) has made it possible to detect overlapping clones allowing the reconstruction of a putative genomic RNA of 7582 nucleotides. —Reconstructed sequence is understood to mean the sequence deduced from the alignment of the overlapping clones—. This genomic RNA has the structure R-U5-gag-pol-env-U3-R. A “blastn” interrogation on several databases, with the aid of the reconstructed genome, shows that a large quantity of related genomic sequences (DNA) exist in the human genome. About 400 sequences have been identified in GenBank (cf FIG. 3) and more than 200 sequences in the EST (Expressed Sequence Tag) library, the majority as antisense. These sequences are found on several chromosomes, in particular chromosomes 5, 7, 14, 16, 21, 22, X, with a high apparent concentration of LTR on the X chromosome.

The reconstructed sequence (mRNA) is integrally contained inside the genomic clone RG083M05 (gb AC00064) (9.6 kb), and exhibits 96% similarity with two discontinuous regions of this clone which also contains repeat regions at each end. The alignment of the experimental sequences corresponding to the 5′ and 3′ regions of the reconstructed genomic RNA with the DNA of the RG083M05 clone has made it possible to deduce an LTR sequence and to identify elements characteristic of retroviruses, in particular those involved in reverse transcription, namely the PBS (Primer Binding Site) downstream of the 5′ LTR and the PPT (PolyPurine Tract) upstream of the 3′ LTR. It is observed that the U3 element is extremely short in comparison with the mammalian type C retroviruses, and comparable in size to the U3 region generally described in the type D retroviruses and the avian retroviruses. The PBS region is homologous to the PBS of the avian retroviruses, suggesting the use of the tRNA^{Trp} as primer for the reverse transcription. Consequently, this new family of HERV is called HERV-W (Human Endogenous RetroVirus).

Phylogenetic analysis in the pol region has shown that the HERV-W family is phylogenetically linked to the ERV-9 and RTVL-H families, and therefore belongs to the family of type I endogenous retroviruses. Phylogenetic analysis of the open reading frame (ORF) of env shows that it is closer to the type D simian retroviruses and the avian reticuloendotheliosis retroviruses than type C mammalian retroviruses, suggesting a C/D chimeric genome structure.

The phylogenetic trees, supported by high “bootstrap” values show that the ERV-9 and HERV-W families are derived from two waves of independent insertions. Thus, the active element(s) at the origin of the HERV-W family is (are) different from that (those) from which the ERV-9 family is derived. Furthermore, the PBS of HERV-W probably uses a tRNA^{Trp} whereas ERV-9 probably uses a tRNA^{Arg}.

Finally, the members of the HERV-W family are expressed in the placenta, whereas the ERV-9 RNAs are not detected in this tissue.

Biological Functions of HERV-W

The expression of HERV-W restricted to the placenta and the long reading frame potentially encoding a retroviral envelope make it possible to propose physiological biological functions whose impairment could be associated with pathologies.

The expression restricted to the placenta suggests that the expression of retroviral and/or nonretroviral genes under the control of the LTRs may be hormone-dependent. These genes may be adjacent, or under the control of isolated LTRs. A pathology may then result from an aberrant expression following the reactivation of a silent LTR by various factors: viral infection (for example by a member of the Herpesvirus family) or local immune activation. A polymorphism at the level of the LTRs could also promote these events.

The envelope of HERV-W could play a fusogenic role, in particular at the level of cellular subtypes of the placenta. An immunosuppressive peptide of this envelope could protect the fetus against attack by the maternal immune system. Finally, by a mechanism of saturation of receptors, the envelope of HERV-W could play a protective role against exogenous retroviral infections. The impairment of local cellular immunity may result from an immunostimulatory signal carried by the envelope. This effect may be linked to a region carrying a superantigen activity, or to the immunosuppressive region which would become immunostimulatory following either a polymorphism or a dose-effect (overexpression).

Verification of these implications and understanding of the consequences linked to an impairment of the biological functions of the endogenous LTRs or the retroviral envelope may lead to the establishment of methods of diagnosis or of monitoring:

of states of pathological pregnancy or of unsuccessful pregnancy,

of autoimmune diseases such as multiple sclerosis or rheumatoid arthritis.

In accordance with the present invention, there has been discovered, in the endogenous state, a new nucleic material, stated explicitly and described below, having the organization of a retrovirus, and capable of being correlated with an autoimmune disease, or a pathology which is associated with it, with a pathological pregnancy or an unsuccessful pregnancy.

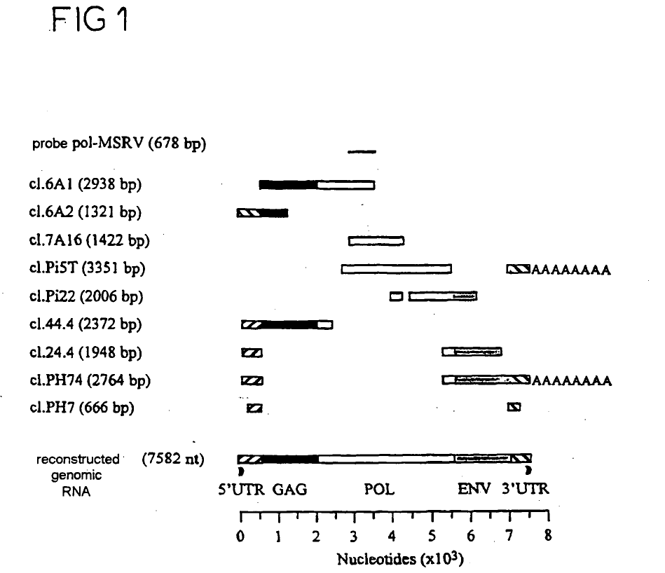

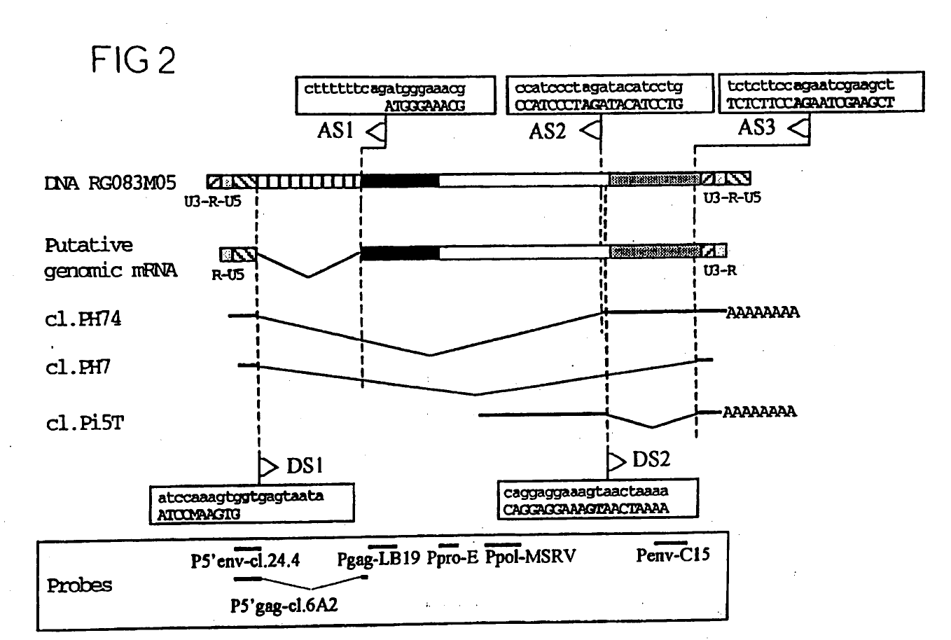

The nucleic material according to the present invention, in mRNA form, represents about 8 Kb; it is represented in FIG. 1 and is described by SEQ ID NO: 11, and is represented in FIG. 2 in the form of genomic DNA.

The expression “of retroviral type” is understood to mean the characteristic according to which the nucleic material considered comprises one or more nucleotide sequences related to the organization of a retrovirus, and/or to its functional or coding sequences.

This reference nucleic material is related to a human endogenous retrovirus, designated by the expression HERV-W. Consequently, it may be obtained by any appropriate technique for screening any library of human DNA, or of placental cDNA, as shown below, in particular with nucleic primers or probes synthesized so as to hybridize with all or part of SEQ ID NO.: 11.

The present invention also relates to any nucleic or peptide product, obtained or derived from the reference nucleic material, according to SEQ ID NO: 11.

And finally, the invention relates to the various correlations which may be made between the abovementioned nucleic material, and/or its derived products, with any autoimmune disease and/or a pathology which is associated with it, as well as with cases of pathological pregnancy or of unsuccessful pregnancy.

“Autoimmune” is understood to mean in particular:

multiple sclerosis

rheumatoid arthritis

disseminated lupus erythematosus

insulin-dependent diabetes

and/or pathologies which are associated with them.

The present invention relates, first of all, to a nucleic material of the retroviral genomic type, in isolated or purified state, at least partially functional or nonfunctional.

This material is characterized in that its genome comprises a reference nucleotide sequence chosen from the group including the sequences SEQ ID NOs: 1 to 15, their complementary sequences, and their equivalent sequences, in particular the nucleotide sequences exhibiting, for any sequence of 100 contiguous monomers, at least 50% and preferably at least 70%, for example at least 90% homology with respectively said sequences SEQ ID NOs: 1 to 15.

This material is also characterized in that its genome comprises a reference nucleotide sequence, encoding any polypeptide exhibiting, for any contiguous sequence of at least 30 amino acids, at least 50%, and preferably at least 70% homology with a peptide sequence capable of being encoded by at least a functional part of the reference nucleotide sequence as defined above.

In particular, this material comprises a nucleic fragment inserted between two sequences corresponding respectively to the LTR region and to the gag gene for the retroviral genomic structure, in particular a nucleic fragment consisting of or comprising the sequence SEQ ID NO: 12.

The invention also relates to a nucleic material of the subgenomic retroviral type, consisting of a nucleotide sequence identical to SEQ ID NO: 11, with a deletion as exemplified by the clones cl.PH74 (SEQ ID NO: 7), cl.PH7 (SEQ ID NO: 8) and cl.Pi5T (SEQ ID NO: 9), this deletion resulting or otherwise from a splicing strategy.

The above-defined nucleic material comprises at least one functional nucleotide sequence encoding at least one retroviral protein, and/or at least one regulatory nucleotide sequence.

Next, the invention relates to any nucleotide fragment of at least 100 bases, comprising a nucleotide sequence chosen from the group comprising:

a) all the nucleotide sequences, partial and complete, of a nucleic material as defined above

b) all the nucleotide sequences, partial and complete, of a clone chosen from the group including the clones:

cl.6A2 (SEQ ID NO: 1)

cl.6A1 (SEQ ID NO: 2)

cl.7A16 (SEQ ID NO: 3)

cl.Pi22 (SEQ ID NO: 4)

cl.24.4 (SEQ ID NO: 5)

cl.C4C5 (SEQ ID NO: 6)

cl.PH74 (SEQ ID NO: 7)

cl.PH7 (SEQ ID NO: 8)

cl.Pi5T (SEQ ID NO: 9)

cl.44.4 (SEQ ID NO: 10)

HERV-W (SEQ ID NO: 11)

cl.6A5 (SEQ ID NO: 12)

cl.7A20 (SEQ ID NO: 13)

cl.7A21 (SEQ ID NO: 14)

LTR (SEQ ID NO: 15)

c) the sequences which are respectively complementary to the sequences according to a) and b)

d) the sequences which are respectively equivalent to the sequences according to a) to c), in particular the nucleotide sequences exhibiting, for any sequence of 100 contiguous monomers, at least 50%, and preferably at least 70%, or even better at least 80%, for example at least 90% homology with the sequences a) to c).

The invention also relates to any nucleic probe for the detection of a nucleic material, inserted or otherwise into a nucleic acid, characterized in that it is capable of hybridizing specifically with a nucleic material, as defined above.

Such a probe comprises a marker or otherwise.

The invention also relates to a nucleic primer for the amplification by polymerization of an RNA or of a DNA, characterized in that it comprises a nucleotide sequence capable of hybridizing specifically with a nucleic material or a nucleic fragment, as defined above.

By way of example, a nucleic probe or nucleic primer according to the invention is characterized in that it consists of a nucleotide sequence chosen from the group including SEQ ID NOs: 16 to 28.

The invention also relates to any RNA or DNA, and in particular a replication vector, comprising a nucleotide fragment, as defined above.

The invention also relates to any peptide encoded by any open reading frame belonging to a nucleotide fragment, as defined above, in particular polypeptide, for example oligopeptide forming an antigenic determinant recognized by sera from patients affected by an autoimmune disease, or a pathology which is associated with it, or from patients having a pathological pregnancy or an unsuccessful pregnancy.

By way of example, this polypeptide is encoded by a nucleotide fragment comprising an open reading frame encoding one or more retroviral ENV proteins.

Finally, the invention relates to:

the use of a nucleic material, or of a nucleotide fragment, or of a peptide defined above, as previously defined, as molecular marker for an autoimmune disease or for a pathology which is associated with it, for pathological pregnancy or unsuccessful pregnancy;

the use of a nucleic material, or of a nucleotide fragment, as defined above, as chromosomal marker for susceptibility to an autoimmune disease or for a pathology which is associated with it, or for a risk of a pathological pregnancy or of an unsuccessful pregnancy;

the use of a nucleic material, or of a nucleotide fragment, as defined above, as proximity marker for a gene for susceptibility to an autoimmune disease or to a pathology which is associated with it, or to a risk of a pathological pregnancy or of an unsuccessful pregnancy.

The invention also relates to a method for the molecular labeling of an autoimmune disease or of a pathology which is associated with it, of pathological pregnancy or of unsuccessful pregnancy, characterized in that any nucleotide fragment, as defined above, either in RNA form or in DNA form, is identified and/or quantified in any biological body material, in particular body fluid.

By way of example, according to such a method, cells expressing a nucleotide fragment, as defined above, are detected in said biological body material.

The invention relates to a diagnostic and/or therapeutic application of a nucleic material, of a nucleotide fragment or of a peptide defined above, and as such, another subject of the invention is a diagnostic composition or a therapeutic composition comprising said material, said fragment or said peptide.

Before detailing the invention, various terms used in the description and the claims are now defined:

human virus is understood to mean a virus capable of infecting or of being harbored by a human being,

taking into account all the natural or induced variations and/or recombinations which may be encountered in the practical implementation of the present invention, the subjects thereof, defined above and in the claims, have been expressed comprising the equivalents or derivatives of the different biological materials defined below, in particular the homologous nucleotide or peptide sequences,

the variant of a virus or of a pathogenic and/or infective agent according to the invention comprises at least one antigen recognized by at least one antibody directed against at least one corresponding antigen of said virus and/or of said pathogenic and/or infective agent, and/or a genome of which any part is detected by at least one hybridization probe, and/or at least one nucleotide amplification primer specific for said virus and/or pathogenic and/or infective agent, in particular a genome belonging to the HERV-W family, under determined hybridization conditions well known to persons skilled in the art,

according to the invention, a nucleotide fragment or an oligonucleotide or a polynucleotide is a stretch of monomers, or a biopolymer, characterized by the sequence, informational or otherwise, of the natural nucleic acids, capable of hybridizing with any other nucleotide fragment under predetermined conditions, it being possible for the stretch to contain monomers of different chemical structures and to be obtained from a natural nucleic acid molecule and/or by genetic recombination and/or by chemical synthesis; a nucleotide fragment may be identical to a genomic fragment of an element of the HERV-W family considered by the present invention, in particular a gene for the latter, for example pol or env in the case of said element;

thus, a monomer may be a natural nucleotide of a nucleic acid, whose constituent elements are a sugar, a phosphate group and a nitrogen base; in RNA, the sugar is ribose, in DNA, the sugar is 2-deoxyribose; depending on whether DNA or RNA is involved, the nitrogen base is chosen from adenine, guanine, uracil, cytosine, thymine; or the nucleotide may be modified in at least one of the three constituent elements; by way of example, the modification may take place at the level of the bases, generating modified bases such as inosine, 5-methyl-deoxycytidine, deoxyuridine, 5-(dimethylamino)deoxy-uridine, 2,6-diaminopurine, 5-bromodeoxyuridine and any other modified base promoting hybridization; at the level of the sugar, the modification may consist in the replacement of at least one deoxyribose with a polyamide, and at the level of the phosphate group, the modification may consist in its replacement with esters, in particular chosen from diphosphate, alkyl and arylphosphonate and phosphorothioate esters,

“functional” is understood to mean the characteristic according to which a nucleotide sequence, a nucleic material or a nucleotide fragment comprises an “an informational sequence”,

“informational sequence” is understood to mean any ordered sequence of monomers whose chemical nature and the order in a reference direction, constitute or otherwise a functional information of the same quality as that of the natural nucleic acids, for example a reading frame encoding a protein, a regulatory sequence, a splicing site or a recombination site,

hybridization is understood to mean the process during which, under appropriate operating, in particular, stringency, conditions, two nucleotide fragments, having sufficiently complementary sequences, pair to form a complex, in particular double or triple, structure, preferably in the form of a helix,

a probe comprises a nucleotide fragment synthesized in particular by the chemical or polymerization route, or obtained by enzymatic digestion or cleavage of a longer nucleotide fragment, comprising at least six monomers, advantageously from 10 to 100 monomers, preferably 10 to 30 monomers, and possessing a hybridization specificity under determined conditions; preferably, a probe possessing less than 10 monomers is not used alone, but is used in the presence of other probes equally short in size or otherwise; under certain specific conditions, it may be useful to use probes larger than 100 monomers in size; a probe may in particular be used for diagnostic purposes and it will include for example capture and/or detection probes,

the capture probe may be immobilized on a solid support by any appropriate means, that is to say directly or indirectly, for example by covalence or by passive adsorption,

the detection probe may be labeled by means of a marker chosen in particular from radioactive isotopes, enzymes particularly chosen from peroxidase and alkaline phosphatase and those capable of hydrolyzing a chromogenic, fluorigenic or luminescent substrate, chromophoric chemical compounds, chromogenic, fluorigenic or luminescent compounds, nucleotide base analogs, and biotin,

the probes used for diagnostic purposes of the invention may be used in all the hybridization techniques known to persons skilled in the art, and in particular the techniques termed “DOT-BLOT”, “SOUTHERN BLOT”, “NORTHERN BLOT” which is a technique identical to the “SOUTHERN BLOT” technique but which uses RNA as target, the SANDWICH technique; advantageously, the SANDWICH technique is used in the present invention, comprising a specific capture probe and/or a specific detection probe, it being understood that the capture probe and the detection probe must have a nucleotide sequence which is at least partially different,

any probe according to the present invention may hybridize in vivo or in vitro with RNA and/or with DNA, to block the phenomena of replication, in particular translation and/or transcription, and/or to degrade said DNA and/or RNA,

a primer is a probe comprising at least six monomers, and advantageously from 10 to 30 monomers, possessing a hybridization specificity under determined conditions, for the initiation of an enzymatic polymerization, for example in an amplification technique such as PCR (Polymerase Chain Reaction), in an extension method such as sequencing, in a reverse transcription method and the like,

two nucleotide or peptide sequences are said to be equivalent or derived from each other, or relative to a reference sequence, if functionally the corresponding biopolymers may play substantially the same role, without being identical, in relation to the application or use considered, or in the technique in which they are used; in particular equivalent are two sequences obtained because of the natural variability within the same individual, or the natural diversity from one individual to another within the same species, in particular spontaneous mutation of the species from which they were identified, or induced mutation, as well as two homologous sequences, the homology being defined below,

“variability” is understood to mean any modification, spontaneous or induced, of a sequence, in particular by substitution, and/or insertion, and/or deletion of nucleotides and/or of nucleotide fragments, and/or extension and/or shortening of the sequence at at least one of the ends; an unnatural variability may result from the genetic engineering techniques used, for example from the choice of the synthetic primers, degenerate or otherwise, selected for amplifying a nucleic acid; this variability may result in modifications of any starting sequence, considered as reference, and which may be expressed by a degree of homology relative to said reference sequence,

homology characterizes the degree of identity of two nucleotide or peptide fragments compared; it is measured by the percentage identity which is in particular determined by direct comparison of nucleotide or peptide sequences, relative to reference nucleotide or peptide sequences,

this percentage identity was specifically determined for the nucleotide fragments, in particular clones within the present invention, and obtained from the same individual; by way of nonlimiting example, the lowest percentage identity observed between the different clones from the same individual (cf SEQ ID NOs: 13 and 14) is at least 90% and the lowest percentage identity observed between the different clones of two individuals is at least 80%,

any nucleotide fragment is said to be equivalent to or derived from a reference fragment if it exhibits a nucleotide sequence equivalent to the sequence of the reference fragment; according to the above definition, particularly equivalent to a reference nucleotide fragment are:

(a) any fragment capable of at least partially hybridizing with the complement of the reference fragment,

(b) any fragment whose alignment with the reference fragment leads to identical contiguous bases being identified in a larger number than with any other fragment obtained from another taxonomic group,

(c) any fragment resulting or capable of resulting from the natural variability within the same individual, and from the natural diversity from one individual to another within the same species, from which it is obtained,

(d) any fragment capable of resulting from genetic engineering techniques applied to the reference fragment,

(e) any fragment, containing at least eight contiguous nucleotides, encoding a peptide homologous or identical to the peptide encoded by the reference fragment,

(f) any fragment different from the reference fragment by insertion, deletion, substitution of at least one monomer, extension, or shortening at at least one of its ends; for example, any fragment corresponding to the reference fragment, flanked at at least one of its ends by a nucleotide sequence not encoding a polypeptide,

partial or complete nucleotide sequence of a reference nucleic material is also understood to mean any sequence associated by co-encapsidation, or by coexpression, or recombined with said reference nucleic material,

polypeptide is understood to mean in particular any peptide of at least two amino acids, in particular oligopeptide or a protein, extracted, separated or substantially isolated or synthesized, through the intervention of human hands, in particular those obtained by chemical synthesis, or by expression in a recombinant organism,

polypeptide partially encoded by a nucleotide fragment is understood to mean a polypeptide having at least three amino acids encoded by at least nine contiguous monomers contained in said nucleotide fragment,

an amino acid is said to be analogous to another amino acid when their respective physicochemical characteristics, such as polarity, hydrophobicity and/or basicity, and/or acidity, and/or neutrality, are substantially the same; thus, a leucine is analogous to an isoleucine,

any polypeptide is said to be equivalent to or derived from a reference polypeptide if the compared polypeptides have substantially the same properties, and in particular the same antigenic, immunological, enzymological and/or molecular recognition properties; particularly equivalent to a reference polypeptide is:

(a) any polypeptide possessing a sequence in which at least one amino acid has been substituted with an analogous amino acid;

(b) any polypeptide having an equivalent peptide sequence obtained by natural or induced variation of said reference polypeptide, and/or of the nucleotide fragment encoding said polypeptide,

(c) a mimotope of said reference polypeptide, (d) any polypeptide in whose sequence one or more amino acids of the L series are replaced by an amino acid of the D series, and vice versa,

(e) any polypeptide into whose sequence a modification of the side chains of the amino acids has been introduced, such as for example an acetylation of the amine functions, a carboxylation of the thiol functions, an esterification of the carboxyl functions,

(f) any polypeptide in whose sequence one or more peptide bonds have been modified, such as for example the carba, retro, inverse, retro-inverse, reduced and methyleneoxy bonds,

(g) any polypeptide of which at least one antigen is recognized by an antibody directed against a reference polypeptide,

the percentage identity characterizing the homology between two compared peptide fragments is, according to the present invention, at least 80% and preferably at least 90%.

The expressions relating to order which are used in the present description and the claims, such as “first nucleotide sequence” are not selected to express a particular order, but to define the invention more clearly.

Detection of a substance or agent is understood to mean hereinafter both an identification and a quantification, or a separation or isolation of said substance or of said agent.

The invention will be understood more clearly upon reading the detailed description which follows, made with reference to the appended figures in which:

FIG. 1 represents, on the one hand, the organization of the endogenous retroviral material discovered according to the present invention, in the form of a putative genomic mRNA, and, on the other hand, the location of the clones used according to the present invention, relative to this organization; the scales for length are expressed in Kb; the flanking regions (5′ UTR and 3′ UTR) are indicated in hatched boxes; the regions repeated in these two flanking regions are indicated by black arrows; the regions corresponding to the gag, pol and env genes are indicated in black, white and gray respectively; the position of the Ppol-MSRV probe is indicated;

FIG. 2 represents a possibility of genetic organization (DNA), illustrated by the clone RG083M05, and a splicing strategy linking to this sequence, the experimental clones (mRNA); this figure also shows the splicing sites observed with reference to the retroviral organization; additionally indicated in this figure are:

the location of the probes used (Pgag-LB19, Ppro-E, Ppol-MSRV and Penv-C15);

the splice donor sites (DS1 and DS2) and acceptor sites (AS1 to AS3);

the sequences obtained from the clone RG083M05, in the lower-case boxes, and the sequences derived from experimental placental clones (mRNA), in the upper-case boxes;

the putative ORFs (ORF1, ORF2 and ORF3); and

an insert of 2 Kb present in DNA form but not detected in RNA form, represented in the form of vertical hatches.

The other conventions used in this figure are the same as those for FIG. 1.

FIG. 3 gives a representation of genomic (DNA) clones corresponding to the isolated cDNA clones; indicated in this figure are:

the percentage similarity with respect to the reconstructed genomic RNA (Recons RNA);

the presence of repeat sequences at each end of these genomes (repeats); and

the presence and the size of the open reading frames (ORFs).

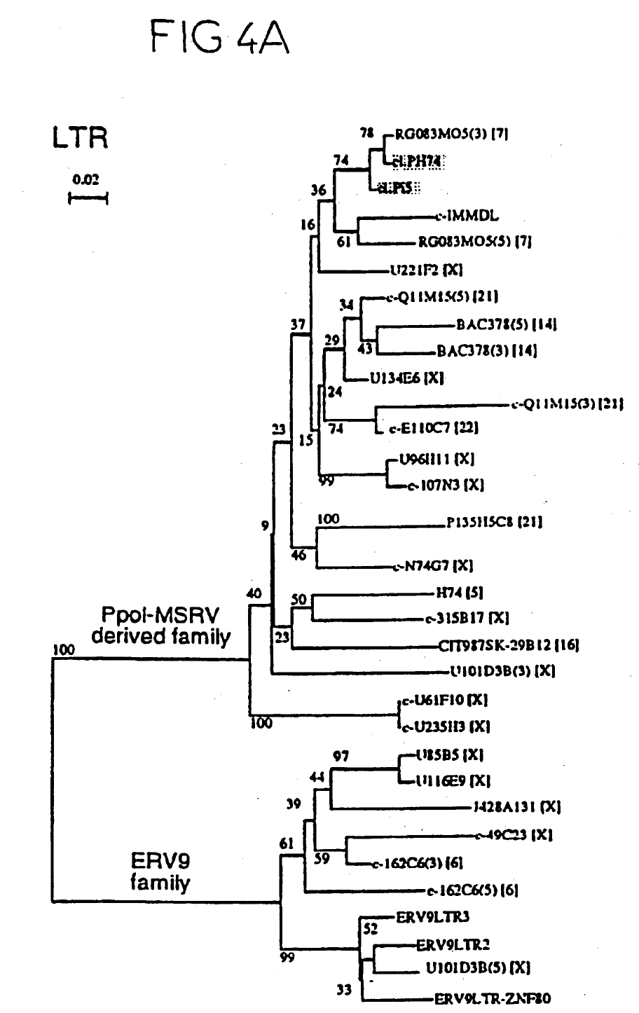

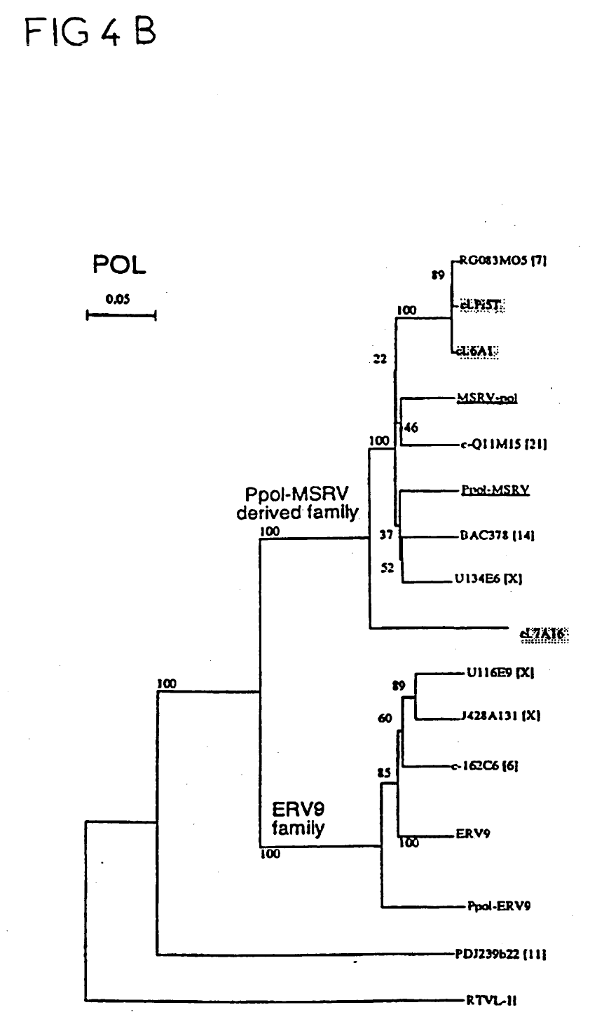

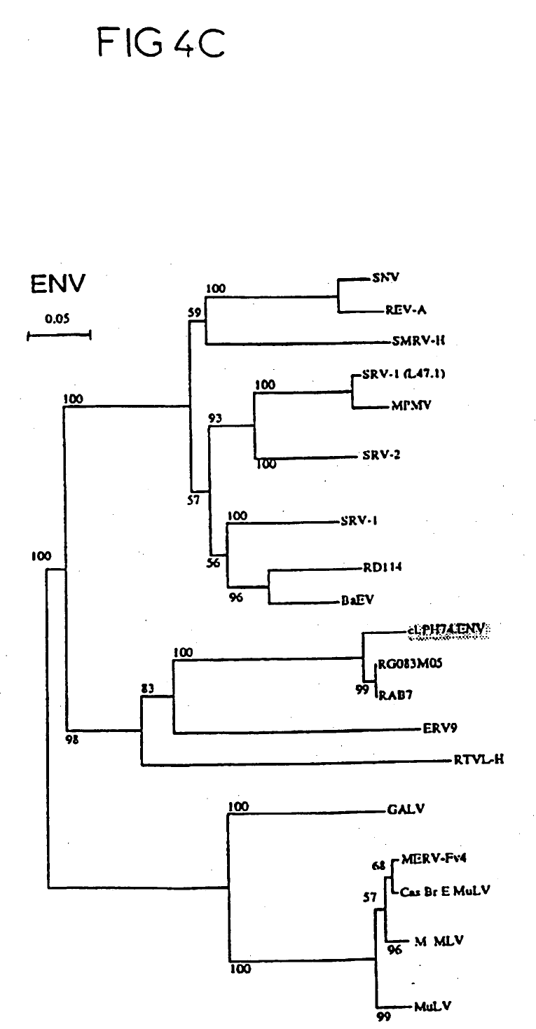

FIG. 4 represents a phylogenetic analysis identifying the HERV-W family.

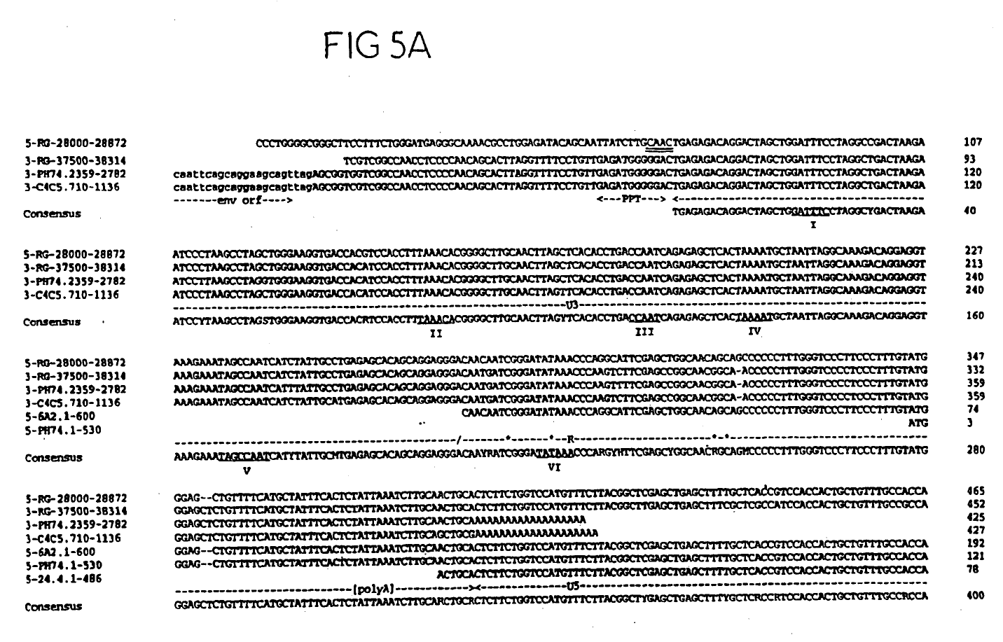

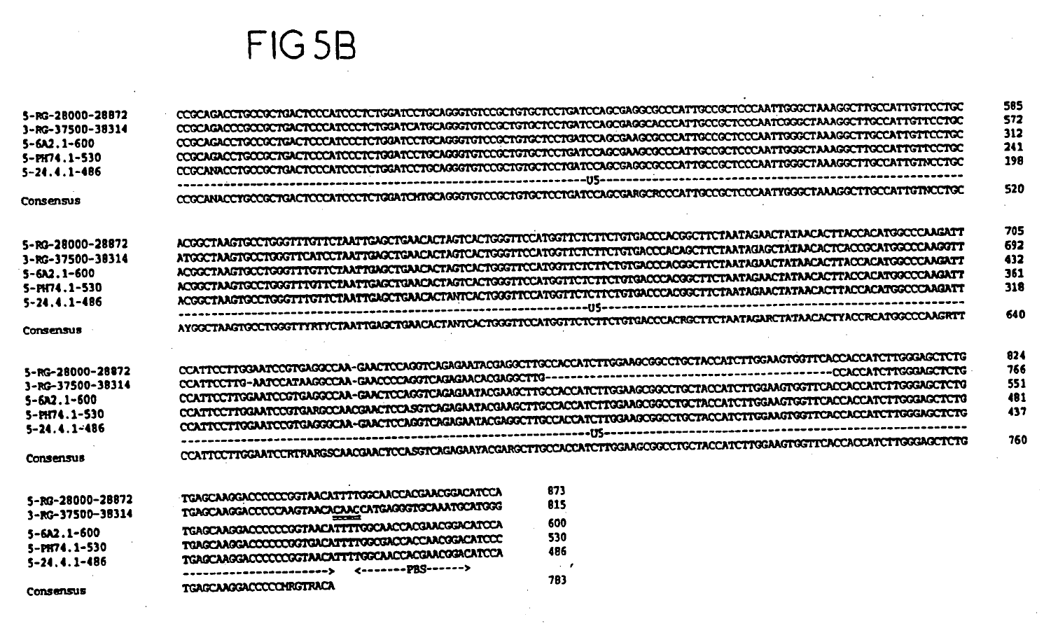

FIG. 5 represents the alignment of the 5′ and 3′ flanking regions of the clone RG083M05 with the terminal 5′ and/or 3′ regions of some placental clones; the CAAC tandem flanking the 3′ and 5′ LTRs is doubly underlined under the DNA sequences, the consensus LTR sequence of 783 bp (base pairs) is indicated under the alignment; the PPT upstream of the 5′ end of LTR and the PBS downstream of the 3′ end of LTR are indicated; the U3R and U5 regions are indicated; the sites corresponding to the binding of the transcription factor are underlined and numbered from 1 to 6; the region 73 to 284 corresponds to the sequence evaluated in “CAT assay”; * corresponds to putative sites for “capping”; [polyA] indicates the polyadenylation signal.

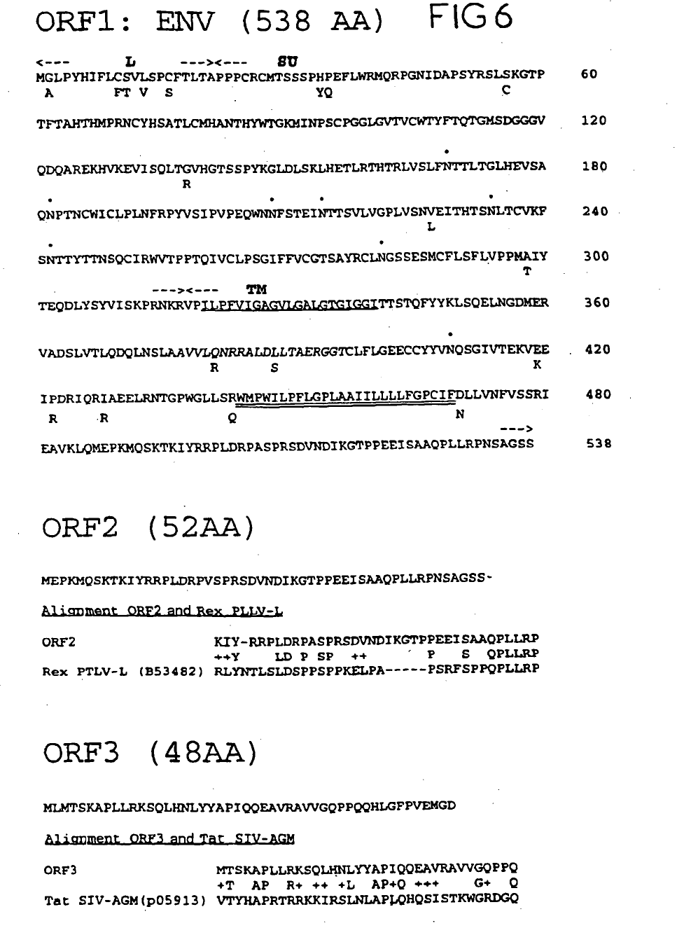

FIG. 6 represents a putative sequence of a HERV-W envelope polypeptide (ORF1) obtained from 3 different placental cDNA clones; the leader peptide (L), the surface protein (SU) and the transmembrane protein (TM) are indicated by arrows; the hydrophobic fusion peptide and the transmembrane carboxy region are underlined by a single line and a double line, respectively; the immunosuppression region is indicated in italics; the potential glycosylation sites are indicated by dots; the divergent amino acids are indicated on the bottom line; FIG. 6 also presents the open reading frames corresponding to ORF2 and ORF3 as described in FIG. 2, and more particularly their homologies with the retroviral regulatory genes.

The nucleic material previously presented explicitly was discovered and characterized at the end of the experimental protocol described below, it being understood that this protocol cannot limit the scope of the present invention and of the accompanying claims.

### EXAMPLE 1

### Isolation and Sequencing of Overlapping cDNA Fragments

The information relating to the organization of HERV-W were obtained by testing a placental cDNA library (Clontech cat#HL5014a) with the probes Ppol-MSRV (SEQ ID NO: 29) and Penv-C15 (SEQ ID NO: 31) (cf Example 8), and then performing a “gene walking” technique with the aid of the new sequences obtained. The experiments were carried out with reference to the recommendations of the supplier of the library. PCR amplifications on DNA were also exploited in order to understand this organization.

A number of clones were selected and sequenced, cf FIG. 1:

clone cl.6A2 (SEQ ID NO: 1): untranslated 5′ region of HERV-W and part of gag

clone cl.6A1 (SEQ ID NO: 2): gag and part of pol

clone cl.7A16 (SEQ ID NO: 3): 3′ region of pol

clone cl.Pi22 (SEQ ID NO: 4): 3′ region of pol and beginning of env

clone cl.24.4 (SEQ ID NO: 5): spliced RNA comprising part of the untranslated 5′ region of HERV-W, the end of pol and the 5′ region of env

clone cl.C4C5. (SEQ ID NO: 6): end of env and untranslated 3′ region of HERV-W

clone cl.PH74 (SEQ ID NO: 7): subgenomic RNA: untranslated 5′ region of HERV-W, end of pol, env and untranslated 3′ region of HERV-W

clone cl.PH7 (SEQ ID NO: 8): multispliced RNA: untranslated 5′ region of HERV-W, end of env and untranslated 3′ region of HERV-W.

clone cl.Pi5T (SEQ ID NO: 9): partial pol gene and U3-R region

clone cl.44.4 (SEQ ID NO: 10): R-U5 region, gag gene and partial pol gene.

With the aid of these clones, by carrying out sequence alignments, a model of complete sequence of HERV-W was produced. The spliced RNAs were identified as well as the. potential splice donor and acceptor sites. This set of information is shown in FIG. 2. Through a study of similarity with existing retroviruses, the LTR, gag, pol and env entities were defined.

The putative genetic organization of HERV-W in
RNA form is the following (SEQ ID NO: 11):

| | |
|---|---|
| gene | 1 . . . 7582 |
| location of the | cl.6A2 (1321 bp) 1-1325; |
| clones on the | cl.PH74 (535 + 2229 = 2764 bp) 72-606 and 5353-7582; |
| reconstructed | cl.24.4 (491 + 1457 = 1948 bp); 115-606 and |
| genomic RNA | 5353-6810; |
| sequence | cl.44.4 (2372 bp) 115-2496; |
| | cl.PH7 (369 + 297 = 666 bp) 237-606 and 7017-7313; |
| | cl.6A1 (2938 bp) 586-3559.; |
| | cl.Pi5T (2785 + 566 = 3351 bp) 2747-5557 and |
| | 7017-7582; |
| | cl.7A16 (1422 bp) 2908-4337; |
| | cl.Pi22 (317 + 1689 = 2006 bp) 3957-4273 and |
| | 4476-6168; |
| | cl.C4C5 (1116 bp) 6467-7582 |
| 5′LTR | 1 . . . 120 |
| | /note=“R of 5′LTR (5′ end uncertain” |
| | 121 . . . 575 |
| | /note=“U5 of 5′LTR” |
| various | 579 . . . 596 |
| | /note=“PBS primer binding site for tRNA-W” |
| various | 606 |
| | /note=“splice junction (splice donor site |
| | ATCCAAAGTG-GTGAGTAATA and splice acceptor |
| | site CTTTTTTCAG-ATGGGAAACG clone RG083M05, |
| | GenBank accession AC000064)” |
| various | 5353 |
| | /note=“splice acceptor site for ORF1 (env)” |
| various | 5560 |
| | /note=“splice donor site” |
| ORF | 5581 . . . 7194 |
| | /note=“ORF1 env 538 AA” |
| | /product-=“envelope” |
| various | 7017 |
| | /note=“splice acceptor site for ORF2 and |
| | ORF3” |
| ORF | 7039 . . . 7194 |
| | /note=“ORF2 52 AA” |
| ORF | 7112 . . . 7255 |
| | /note=“ORF3 48 AA” |
| various | 7244 . . . 7254 |
| | /note=“PPT polypurine tract” |
| 3′LTR | 7256 . . . 7582 |
| | /note-=“U3-R of 3′ LTR (U3-R junction |
| | indeterminate) |
| various | 7563 . . . 7569 |
| | polyadenylation signal |

### EXAMPLE 2

### Identification of Genomic (DNA) Clones Corresponding to the Isolated DNA Clones

A “blastn” interrogation of several databases, with the aid of the reconstructed genome, shows that a large quantity of related sequences exist in the human genome. About 400 sequences were identified in GenBank and more than 200 sequences in the EST library, and the majority as antisense. The 4 sequences most significant in size and in similarity, illustrated in FIG. 3, are the following genomic (DNA) clones:

the human clone RG083M05 (gb AC000064) whose chromosomal location is 7q21-7q22,

the human clone BAC378 (gb U85196, gb AE000660) corresponding to the alpha delta locus of the T cell receptor, located in 14q11-12,

the human cosmid Q11M15 (gb AF045450) corresponding to the 21q22.3 region of chromosome 21,

the cosmid U134E6 (embl Z83850) on chromosome Xq22.

The location of the aligned regions for each of the clones is indicated and the affiliation to a chromosome is indicated in square brackets. The percentage similarity (without broad deletions) between the 4 sequences and the reconstructed genomic RNA is indicated, as well as the presence of repeat sequences at each end of the genome and the size of the largest reading frames (ORF). Repeat sequences are found at the ends of 3 of these clones. The reconstructed sequence is integrally contained inside the clone RG083M05 (9.6 Kb) and exhibits a 96% similarity. However, the clone RG083M05 exhibits an insert of 2 Kb situated immediately downstream of the untranslated 5′ region (5′ UTR). This insert is also found in two other genomic clones which exhibit a deletion of 2.3 Kb immediately upstream of the untranslated 3′ region (3′ UTR). No clone contains the three functional reading frames (ORFs) gag, pol and env. The clone RG083M05 shows an ORF of 538 amino acids (AA) corresponding to a whole envelope. The cosmid Q11M15 contains two large contiguous ORFs of 413 AA (frame 0) and 305 AA (frame +1) corresponding to a truncated pol polyprotein.

### EXAMPLE 3

### Phylogenetic Analysis

A phylogenetic analysis was carried out at the level of the nucleic acids on 11 different subregions of the reconstructed genomic RNA, and at the protein level on 2 different subregions of env. All the trees obtained exhibit the same topology regardless of the region studied. This is illustrated in FIG. 4 at the level of the nucleic acids in the most conserved LTR and pol regions between the sequences obtained and ERV-9 and RTLV-H. The trees clearly show that the experimental sequences describe a new family distinct from ERV-9 and very distinct from RTLV-H as underlined by the “bootstrap” analysis. These sequences are found on several chromosomes, in particular chromosomes 5, 7, 14, 16, 21, 22 and X with a high apparent concentration of LTR on the X chromosome.

Comparison at the protein level between the most conserved regions of the retroviral env proteins shows that the HERV-W family is closer to the type D simian retroviruses and the avian reticuloendotheliosis retroviruses than the type C mammalian retroviruses.

This suggests a C/D chimeric genomic structure.

### EXAMPLE 4

### Identification of the LTR, PPT and PBS Elements

The reconstructed sequence (RNA) is integrally contained inside the genomic clone RG083M05 (9.6 Kb) and exhibits a 96% similarity with two discontinuous regions of this clone which also contains repeat regions at each end. The alignment of the experimental sequences corresponding to the 5′ and 3′ regions of the genomic RNA reconstructed with the DNA of the clone RG083M05 [5′(5-RG-28000-28872) and 3′(3-RG-37500-38314)] made it possible to deduce an LTR sequence and to identify elements characteristic of the retroviruses, in particular those involved in the reverse transcription, namely PBS downstream of the 5′ LTR and the PPT upstream of the 3′ LTR (cf FIG. 5). It is observed that the U3 element is extremely short in comparison with that observed in the mammalian type C retroviruses, and is comparable in size to the U3 region generally described in the type D retroviruses and the avian retroviruses. The region corresponding to bases 2364 to 2720 of the clone cl.PH74 (SEQ ID NO: 7) was amplified by PCR and subcloned into the vector pCAT3 (Promega) in order to carry out the evaluation of the promoter activity. A significant activity was found in HeLa cells by the so-called “CAT assay” method showing the functionality of the promoter sequence of the LTR.

The PBS region is homologous to the PBS of the avian retroviruses.

### EXAMPLE 5

### Genetic Organization and Regulation of Expression

Organization in DNA Form

PCR amplifications were carried out on whole HERV-W clones recovered on human genomic library (see Example 1 for the mode of production), using the following oligonucleotide pairs:

U5 4992 (SEQ ID NO: 16), GAG 4619 (SEQ ID NO: 17)

GAG 4782 (SEQ ID NO: 18), POL 3167 (SEQ ID NO: 19)

POL 3390 (SEQ ID NO: 20), POL 5144 (SEQ ID NO: 21)

POL 5145 (SEQ ID NO: 22), U5 4991 (SEQ ID NO: 23).

The PCRs were carried out under the following conditions:

oligonucleotides at the concentration of 0.33 microMolar

TAQ polymerase buffer Boerhinger 1X

0.5 unit of TAQ polymerase Boerhinger

mixture of DNTP at 0.25 mM each

0.5 mg of human DNA

final volume 100 ml

PCR conditions (95° C., 5 min)×1, (95° C., 30 sec+54° C., 30 sec+72° C. 3 min)×35.

The PCR products were then deposited on 1% agarose gel to be analyzed after migration. The set of PCRs gives amplification fragments of the expected size, except for the LTR-4991-gag-4619 PCR which gives a fragment of size greater by about 2 Kb relative to the expected size (deduced from cDNAs from the placental library). The reconstruction of HERV-W in endogenous DNA form therefore represents an entity of about 10 Kb.

After cloning, sequencing and analysis of the PCR-4992 gag-4619, the presence of a region of insertion is observed between LTR and gag of SEQ ID NO: 12 (clone cl.6A5). This region does not correspond to an untranslated traditional region of a retrovirus: no ψ or PBS region.

The products of PCR pol-3390, pol-5144 were also cloned and two of the clones obtained were sequenced. The result of these sequences is given by the clones cl.7A20 (SEQ ID NO: 13) and cl.7A21 (SEQ ID NO: 14). Comparison of these two nucleotide sequences gives a score of 90% homology for the relevant region, thus showing the variability of HERV-W in the same individual.

HERV-W in DNA form is proposed in FIG. 2.

General Organization: Transcription Process

The various cDNA clones having been obtained, results acquired in PCR on DNA, there is deduced:

a DNA organization of 10 Kb possessing an insertion sequence of 2 Kb between LTR and gag.

The result of PCR on DNA showing the presence of an insert of 2 Kb between the LTR and gag regions suggests that the cDNAs isolated from the placenta are obtained from the expression of a genome of the RGO83M05 type.

an RNA organization of 8 Kb resulting from a transcription of 10 Kb followed by a splicing between LTR and gag making it possible to restore a continuity FR (Flanking Region) 5′ gag, and thus giving an RNA of 8 Kb as identified in Northern blotting.

The probes gag (Pgag-LB19, SEQ ID NO: 30) and protease (Ppro-E, SEQ ID NO: 32) reveal an RNA having a size close to 8 Kb, the probe Penv-C15 (SEQ ID NO: 31) reveals, in addition, an RNA close to 3.1 Kb. Two probes defined in the untranslated 5′ region, obtained by screening of the cDNA library reported above (probe P5′-gag-cl.6A2 derived from the clone cl.6A2 and probe P5′-env-cl.24.4 derived from the clone cl.24.4) reveal the preceding two. RNAs and an RNA of about 1.3 Kb. This distribution of the RNAs is typical of complex retrovirus transcripts: a genomic RNA encoding gag-pro-pol, a subgenomic RNA encoding the envelope, and one or more multispliced RNAs potentially encoding regulatory genes.

The half-life of such an RNA (LTR-R-U5-Insertion-GAG-POL-ENV-U3-R-HERV-W) is probably very short, because no RNA of 10 Kb is detected in Northern blotting. By analyzing and comparing sequences, the potential splice donor sites (DS1 and DS2) and acceptor sites were defined and described in FIG. 2.

### EXAMPLE 6

### Transcription in Healthy Tissues

Various healthy human tissues were tested by the Northern-blot technique (Human Multiple Tissue Northern Blot, Clontech cat# 7760-1), with the aid of the probes Ppol-MSRV (SEQ ID NO: 29), Pgag-LB19 (SEQ ID NO: 30), Penv-C15 (SEQ ID NO: 31), Ppro-E (SEQ ID NO: 32), P5′-gag-cl.6A2 and P5′-env-cl.24.4, labeled as described in Example 1. The experiments were carried out following the recommendations of the manufacturers, and the autoradiographs were exposed for 5 days. Analysis of the results reveals transcription products only in the placenta, and in none of the other human tissues tested (heart, brain, lungs, liver, skeletal muscle, kidney and pancreas).

Using an RNA Dot-Blot technique (Clontech: Human RNA Master Blot Cat# 7770-1), and using the experimental protocol recommended by the manufacturer, about forty other tissues, including fetal tissues, were tested: only the placenta gives a specific response after hybridization with the probes Pgag-LB19 (SEQ ID NO: 30) and Penv-C15 (SEQ ID NO: 31).

It is observed that a signal is observed in the kidney in RNA Dot-Blot, which is infirmed by the Northern-blot analysis.

### EXAMPLE 7

### Identification of an mRNA Encoding an Envelope and the Means for Detecting it specifically

The screening of a placental cDNA library with the aid of a probe defined in the untranslated 5′ region made it possible to isolate a cDNA defined by an untranslated 5′ region (5′ NTR), a splicing junction, a coding sequence, an untranslated 3′ region (3′ NTR) and a polyadenylated tail, cl.PH74 (SEQ ID NO: 7). This clone corresponds to a spliced RNA encoding an envelope. By comparing sequences between this cDNA and the endogenous HERV-W model proposed according to FIG. 2, a splicing junction is identified on the mRNA, a splicing junction placing in continuity the 5′ NTR region and the env gene, leading to the production of a spliced subgenomic RNA encoding the envelope gene. This information made it possible to define an oligonucleotide specific for this mRNA by choosing a location situated on the splicing site (Oligo 5307, according to SEQ ID NO: 24).

The identification of this joining region makes it possible to establish a method of discriminating between endogenous retroviral RNA and DNA, using, in a PCR, an oligonucleotide defined on this joining region, in particular an oligonucleotide chosen from the env gene (Oligo 4986, according to SEQ ID NO: 25).

The PCRs were carried out under the following conditions:

oligonucleotides at the concentration of 0.33 microMolar

TAQ polymerase buffer Boerhinger 1X

0.5 unit of TAQ polymerase Boerhinger

mixture of dNTP at 0.25 mM each

0.5 mg of human DNA

final volume 100 ml

On 10 different DNAs tested, this type of PCR did not make it possible to obtain amplification products. On the other hand, on cDNA derived from placental RNA or from cells expressing HERV-W, this PCR gives an amplification product. This result therefore confirms the specifically RNA nature of this subgenomic fragment.

### EXAMPLE 8

### Identification of Coding Sequences Contained in a Specific mRNA

The splicing strategy described in Example 5 is compatible with the presence of three reading frames ORF1 (SEQ ID NO: 33), ORF2 (SEQ ID NO: 34) and ORF3 (SEQ ID NO: 35) (cf FIG. 6).

The screening of a placental cDNA library made it possible to isolate a cDNA (SEQ ID NO: 7, cl.PH74) defined by an untranslated 5′ region (5′ NTR), a splicing junction, a coding sequence, an untranslated 3′ region (3′ NTR) and a polyadenylated tail. The coding sequence is 538 amino acids (SEQ ID NO: 33). The analyses carried out on databanks make it possible to identify characteristics of a complete retroviral envelope: initiation of translation of an envelope polyprotein, of a highly hydrophobic leader peptide of about 21 amino acids, of a surface protein SU, of a transmembrane protein TM. These two protein entities exhibit different potential glycosylation sites. An immunosuppressive region is identified within the TM protein.

22 bp and 95 bp upstream of the splice acceptor site, two initiation codons were respectively found which were capable of directing the synthesis of 52 AA (ORF2, SEQ ID NO: 34) and of 48 AA (ORF3, SEQ ID NO: 35). ORF2 consists of part of the carboxy-terminal end of env and ORF3 corresponds to a different but overlapping translation.

No significant homology was found by “blast” interrogation. However, an LFASTA interrogation in a sub-databank limited to the Retroviridae, ORF2 and ORF3 showed a percentage identity of 35% with, respectively, Rex of the human and primate lymphotropic T virus, and with Tat of the simian immunodeficiency virus.

### EXAMPLE 9

### Complexity of the HERV-W Family

The number of copies present in the human genome of each of the sequences is evaluated by a Dot-Blot technique, with the aid of the probes Pgag-LB19 (SEQ ID NO: 30), Ppro-E (SEQ ID NO: 32) and Penv-C15 (SEQ ID NO: 31).

Each of the probes is denatured and deposited on a Hybond N+ membrane in an amount of 2.5, 5, 10, 25, 50, 100 pg per deposit. 0.5 mg of human DNA are also deposited on the same membrane. The membranes are dried for 2 hours under vacuum at 80° C. The membranes are then hybridized with the deposited probe. The techniques for labeling the probes, for hybridization and for washing the membranes are the same as for the Southern blotting. After autoradiography of the membranes, levels of signal intensity which are proportional to the deposits on the membrane are observed. After cutting out the hybridization zones, scintillation counting is carried out. By comparison between the dilution series for the probe deposited on the membrane and the result obtained with the human DNA, it is possible to evaluate the number of copies per haploid genome of each of the regions covered by the probes:

the number of endogenous gag is evaluated from 56 to 112 copies (76)

the number of endogenous protease is evaluated from 166 to 334 copies (260)

the number of endogenous env is evaluated at less than 52 copies (13).

The screening of 106 clones of a human placental DNA library (Clontech cat# H15014b) made it possible to count 144 clones recognized by the probe Pgag-LB19, and 64 clones recognized by the probe Penv-C15. 13 clones hybridized conjointly with the probes Penv-C15 and Pgag-LB19 were isolated, confirming the presence of several copies of a genome possessing both gag and env, without consideration of functionality.

The nucleic material, the nucleotide sequences and the peptides or proteins which may be expressed by said materials and sequences may be used to detect, predict, treat and monitor any autoimmune disease, and the pathologies which are associated with it, as well as in cases of pathological pregnancy or of unsuccessful pregnancy.

Indeed, the objective and experimental data make it possible to link retrovirus and autoimmune diseases and retrovirus and pregnancy disorders:

(1) common mechanisms are used in the retroviral pathologies and in autoimmune diseases (presence of autoantibodies, of immune complexes, cellular infiltration of certain tissues, neurological disorders).

(2) pathological disorders comparable to certain autoimmune diseases appear during infections with HIV and HTLV retroviruses (sjögren syndrome, disseminated lupus erythematosus, rheumatoid arthritis and the like).

(3) a reverse transcriptase activity was detected and retroviral-type particles were observed in the cell culture supernatants of patients suffering from multiple sclerosis (Perron et al., Res. Virol. 1989; 140: 551-561/Lancet 1991; 337: 862-863/Res. Virol. 1992; 143: 337-350) or from rheumatoid arthritis.

(4) autoimmune or chronic inflammatory animal pathologies are linked to endogenous retroviruses; some of them are used as animal models of human diseases (insulin-dependent diabetes, disseminated lupus erythematosus)

(5) significant levels of endogenous anti-retrovirus antibodies have been described in the context of autoimmune, systemic or inflammatory diseases; other data of this nature were communicated by several authors at the IVth European meeting on endogenous retroviruses (Uppsala, October 1996). According to Venables (communiques of the IVth European meeting on endogenous retroviruses, Uppsala, October 1996), a significantly high level of anti-HERV-H antibodies are found during pregnancy but also in the context of various autoimmune disorders such as Sjögren syndrome, disseminated lupus erythematosus or rheumatoid arthritis, without, however, any proof of its direct involvement being provided up until now.

The involvement of the retroviruses in the autoimmune phenomenon remains compatible with the multifactorial character of the autoimmune, systemic or inflammatory diseases which confront genetic, hormonal, environmental and infectious factors.

The particles observed in the cell culture supernatants from patients suffering from multiple sclerosis (Perron et al., Res. Virol. 1989; 140: 551-561/Lancet 1991; 337: 862-863/Res. Virol. 1992; 143: 337-350) or from rheumatoid arthritis (unpublished data) may result from the expression: (i) of an endogenous retrovirus competent for replication, (ii) of several defective endogenous retroviruses cooperating by a phenomenon of transcomplementation or (iii) of an exogenous retrovirus.

All these observations make it possible to use and consider the above-described biological material as marker for an autoimmune disease or for pregnancy disorders.

In particular, the following labeling techniques are considered:

screening of the human genome with high-stringency hybridization probes derived from the nucleic material described above,

direct amplification of genomic DNA by PCR, using primers specific for the region considered

analysis of the flanking regions of foreign cellular genes.

What is claimed is:

1. A nucleic material, in an isolated or purified state, comprising a nucleotide sequence selected from the group consisting of sequences of SEQ ID NOs: 1 to 15, their complementary sequences, and sequences that exhibit for every sequence of 100 contiguous monomers at least 70% homology with said sequences of SEQ ID NOs: 1 to 15, respectively.

2. A nucleic material, in an isolated or purified state, comprising a nucleotide sequence, encoding any polypeptide exhibiting, for every contiguous sequence of at least 30 amino acids, at least 80% identity with a peptide sequence encoded by at least a functional part of a nucleotide sequence selected from the group consisting of sequences of SEQ ID NOs: 1 to 15 and their complementary sequences.

3. The nucleic material according to claim 1, comprising a nucleic fragment inserted between two sequences corresponding respectively to the LTR region and to the gag gene for a retroviral genomic structure.

4. A nucleic material consisting of a nucleotide sequence identical to SEQ ID NO: 11, with at least one deletion.

5. A nucleic material according to claim 1, comprising at least one functional nucleotide sequence encoding at least one retroviral protein.

6. A nucleic material according to claim 1, comprising at least one regulatory nucleotide sequence.

7. A nucleotide fragment comprising a nucleotide sequence selected from the group consisting of:
(a) a nucleotide sequence of at least 100 bases of a clone selected from the group consisting of:
cl.6A2 (SEQ ID NO: 1),
cl.6A1 (SEQ ID NO: 2),
cl.7A16 (SEQ ID NO: 3),
cl.Pi22 (SEQ ID NO: 4),
cl.24.4 (SEQ ID NO: 5),
cl.C4C5 (SEQ ID NO: 6),
cl.PH74 (SEQ ID NO: 7),
cl.PH7 (SEQ ID NO: 8),
cl.Pi5T (SEQ ID NO: 9),
cl.44.4 (SEQ ID NO: 10),
HERV-W (SEQ ID NO: 11),
cl.6A5 (SEQ ID NO: 12),
cl.7A20 (SEQ ID NO: 13),
cl.7A21 (SEQ ID NO: 14), and
LTR (SEQ ID NO: 15);
(b) sequences which are respectively complementary to the sequences according to (a); and
(c) equivalent sequences which have respectively at least 50% homology to the sequences according to (a) and (b).

8. A nucleic probe for the detection of a nucleic material, wherein said nucleic probe hybridizes under highly stringent conditions with the nucleotide sequence of the nucleic material according to claim 1.

9. A probe according to claim 8, comprising a label.

10. A nucleic primer for the amplification by polymerization of an RNA or of a DNA, comprising a nucleotide sequence that hybridizes under highly stringent conditions with the nucleotide sequence of the nucleic material according to claim 1.

11. A nucleic probe or nucleic primer, comprising a nucleotide sequence selected from the group consisting of SEQ ID NOs: 16 to 28.

12. An RNA or DNA, comprising a nucleotide fragment according to claim 7.

13. The nucleic probe according to claim 8, wherein said probe contains at least 6 monomers.

14. The nucleic probe according to claim 13, wherein said probe contains no more than 100 monomers.

15. The nucleic probe according to claim 13, wherein said probe contains at least 6 contiguous monomers of a sequence selected from the group consisting of SEQ ID NOs: 1-15 and their complementary sequences.

16. The nucleic probe according to claim 8, wherein said probe has at least 70% homology with a sequence selected from the group consisting of SEQ ID NOs: 1-15 and their complementary sequences.

17. The nucleic probe according to claim 16, wherein said probe has at least 90% homology with a sequence selected from the group consisting of SEQ ID NOs: 1-15 and their complementary sequences.

18. A method for the molecular labeling of at least one member selected from the group consisting of an autoimmune disease, a pathology associated with an autoimmune disease, a pathological pregnancy, and an unsuccessful pregnancy, said method comprising:
at least one of identifying and quantifying any nucleotide fragment according to claim 7 in any biological body material.

19. The method according to claim 18, further comprising:
detecting cells expressing the nucleotide fragment in said biological body material.

20. A diagnostic composition comprising a nucleic material according to claim 1.

21. A method of diagnosing an autoimmune disease, a pathology associated with an autoimmune disease, a pathological pregnancy, or an unsuccessful pregnancy, said method comprising:
obtaining a biological sample;
contacting said biological sample with a molecular marker comprising a nucleic material according to claim 1; and
detecting for said molecular marker.

22. A method of diagnosing susceptibility to an autoimmune disease or a pathology associated with an autoimmune disease, a risk of a pathological pregnancy, or a risk of an unsuccessful pregnancy, said method comprising:
obtaining a biological sample;
contacting said biological sample with a chromosomal marker comprising a nucleic material according to claim 1; and
detecting for said chromosomal marker.

23. A method of detecting a gene associated with susceptibility to an autoimmune disease or a pathology associated with an autoimmune disease, a risk of a pathological pregnancy, or a risk of an unsuccessful pregnancy, said method comprising:
obtaining a biological sample;
contacting said biological sample with a proximity marker comprising a nucleic material according to claim 1; and
detecting for said proximity marker.

24. The method of claim 18, wherein said biological body material comprises a body fluid.

25. The nucleic material according to claim 1, wherein said nucleotide sequence exhibits, for every sequence of 100 contiguous monomers, at least 90% homology with said sequences of SEQ ID NOs: 1 to 15, respectively.

26. The nucleic material according to claim 2, wherein said polypeptide exhibits, for every contiguous sequence of at least 30 amino acids, at least 90% identity with a peptide sequence capable of being encoded by at least a functional part of said nucleotide sequence selected from the group consisting of sequences of SEQ ID NOs: 1-15 and their complementary sequences.

27. The nucleic material of the retroviral genomic type according to claim 2, comprising a nucleic fragment inserted between two sequences corresponding respectively to the LTR region and to the gag gene for said retroviral genomic structure.

28. The nucleic material according to claim 27, wherein said nucleic fragment comprises the sequence of SEQ ID NO: 12.

29. The nucleic material according to claim 3, wherein said nucleic fragment comprises the sequence of SEQ ID NO: 12.

30. The nucleic material according to claim 4, wherein said nucleotide sequence comprises a sequence selected from the group consisting of the sequences of SEQ ID NOs: 7, 8 and 9.

31. The nucleic material according to claim 4, comprising at least one functional nucleotide sequence encoding at least one retroviral protein.

32. The nucleic material according to claim 4, comprising at least one regulatory nucleotide sequence.

33. A replication vector, comprising a nucleotide fragment according to claim 7.

34. A nucleotide fragment according to claim 7, wherein said equivalent sequences exhibit at least 70% homology with the sequences according to (a) and (b).

35. A nucleotide fragment according to claim 7, wherein said equivalent sequences exhibit at least 90% homology with the sequences according to (a) and (b).
